(19) United States
(12) Patent Application Publication
(10) Pub. No.: US 2024/0108423 A1
(43) Pub. Date: Apr. 4, 2024

(54) SECURING A DRIVING ELEMENT IN AN INSTRUMENT INTERFACE OF A ROBOTIC SURGICAL INSTRUMENT

(71) Applicant: CMR Surgical Limited, Cambridge (GB)

(72) Inventors: Matthew Knight, Sunnyvale, CA (US); Pedro Riera Martinez, London (GB)

(73) Assignee: CMR Surgical Limited, Cambridge, OT (GB)

(21) Appl. No.: 18/475,962

(22) Filed: Sep. 27, 2023

(30) Foreign Application Priority Data
Sep. 28, 2022 (GB) 2214164.2

(51) Int. Cl. A61B 34/30

(52) U.S. Cl. CPC A61B 34/30; A61B 2034/302; A61B 2034/305; A61B 2034/715

(57) ABSTRACT

A robotic surgical instrument includes an articulation drivable by at least one driving element. Movement of an instrument interface element is transferred to the at least one driving element. A driving element securing member includes at least one tapered side wall and is configured to be coupled to the at least one driving element. A holding member includes an opening configured to receive a fixing member and at least one wall defining a recess having a shape complementary to the shape of the at least one tapered side wall. The fixing member is configured to be received in the opening and apply force to secure the driving element securing member within the recess of the holding member so that the at least one wall of the holding member is in frictional contact with the tapered side wall of the driving element securing member.

### CROSS-REFERENCE TO RELATED APPLICATION

This application claims priority to GB Patent Application No. 2214164.2, filed Sep. 28, 2022, which is incorporated herein by reference in its entirety.

### FIELD OF THE DISCLOSURE

The present disclosure relates to securing at least one driving element (e.g., a cable) in an instrument interface of a robotic surgical instrument.

### BACKGROUND

It is known to use robots for assisting and performing surgery. FIG. 1 illustrates a typical surgical robot 100 which consists of a base 108, an arm 102, and an instrument 105. The base supports the robot, and is itself attached rigidly to, for example, the operating theatre floor, the operating theatre ceiling or a trolley. The arm extends between the base and the instrument. The arm is articulated by means of multiple flexible joints 103 along its length, which are used to locate the surgical instrument in a desired location relative to the patient. The surgical instrument is attached to the distal end 104 of the robot arm. The surgical instrument penetrates the body of the patient 101 at a port 107 so as to access the surgical site. At its distal end, the instrument comprises an end effector 106 for engaging in a medical procedure.

FIG. 2 illustrates a typical robotic surgical instrument 200 for performing robotic laparoscopic surgery. The surgical instrument comprises a base 201 (which may be referred to as an instrument interface) by means of which the surgical instrument connects to the robot arm. A shaft 202 extends between base 201 and articulation 203. Articulation 203 terminates in an end effector 204. In FIG. 2, a pair of serrated jaws are illustrated as the end effector 204. The articulation 203 permits the end effector 204 to move relative to the shaft 202. It is desirable for at least two degrees of freedom to be provided to the motion of the end effector 204 by means of the articulation.

In conventional surgical instruments used as part of a surgical robot as described above the end effectors at the distal end of the instrument are actuated to move by manipulating driving elements (e.g. cables) within the articulation. The cables connect the articulation 203 to the base 201 and may be controlled by the surgical robot arm 102. To actuate the end effectors, portions of the cables at the proximal end of the instrument can be displaced, for example by pulling or pushing the cable, to alter the position of a portion of the cable at the proximal end of the instrument. The portions of the driving elements (e.g. cables) at the proximal end of the instrument (e.g. in the instrument interface 201) can be held by a cable end block that engages the cable and provides a portion that can be gripped more easily than the cable itself. The cable end block, which engages the cable, is then engaged by a body in the instrument interface that allows the cable end block, and thus the cable, to be slidably displaceable to thereby alter the position of the end effector via the articulation 203. The cable is gripped by a cable end block, which in turn is gripped by a body.

However, in some cases it is difficult to provide a secure connection between the cable end block and the cable itself, which may lead to slippage of the cable within the cable end block thus reducing the accuracy of the end effector positioning and moveable range. It is therefore an object of the present disclosure to provide an improved means of securing the cable to the cable end block or equivalent component in order to provide accurate positioning of the end effectors.

Similarly, there are also difficulties in providing a secure connection and engagement between the cable end block and the body in the instrument interface that engages it. In some cases, this can lead to the cable end block becoming displaced relative to the body, therefore reducing the accuracy of the end effector positioning.

Both such instances of relative displacement and slippage require the instrument to be re-zeroed and maintenance to be performed on the articulation to ensure accuracy of the end effector movement. It is therefore an objective of the present application to reduce the occurrence of such instances of slippage and thus the required maintenance burden.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

According to a first aspect of the invention, there is provided a robotic surgical instrument that comprises: an articulation for articulating an end effector, the articulation driveable by at least one driving element; and an instrument interface comprising an instrument interface element for driving the at least one driving element, the instrument interface element movable over a range, the at least one driving element coupled to the instrument interface element such that movement of the instrument interface element is transferred to the at least one driving element; wherein the instrument interface element comprises: a driving element securing member that comprises at least one tapered side wall and is configured to be coupled to the at least one driving element; a fixing member; and a holding member that includes an opening configured to receive the fixing member and at least one wall defining a recess having a shape that is complementary to the shape of the at least one tapered side wall of the driving element securing member; wherein the fixing member is configured to be received in the opening and apply force to secure the driving element securing member within the recess of the holding member so that the at least one wall of the holding member is in frictional contact with the tapered side wall of the driving element securing member.

The robotic surgical instrument as above wherein the driving element securing member may further comprise an opening configured to receive the fixing member; and wherein the fixing member may be configured to be received in the opening of the driving element securing member and the opening of the holding member.

The robotic surgical instrument as above, wherein the recess may include at least two tapered side walls; and the at least two tapered side walls may oppose each other.

The robotic surgical instrument as above, wherein the driving element securing member may be a cable end block, and wherein a first one of the at least two driving elements may be configured to terminate in the cable end block and a second one of the at least two of driving elements is configured to terminate in the cable end block.

The robotic surgical instrument as above, wherein the robotic surgical instrument may comprise a tensioning mechanism for tensioning the at least one driving element; and wherein the tensioning mechanism may comprise a screw adjustment mechanism which couples a pair of drive element securing members together for linearly displacing the pair of drive element securing members with respect to each other.

The robotic surgical instrument as above, wherein the robotic surgical instrument may further comprise an alignment mechanism for setting the displacement position of the instrument interface element to a predetermined alignment position when the end effector has a predetermined configuration; and wherein the screw adjustment mechanism may comprise a screw captive in the first drive element securing member and constrained by the first drive element securing member so as to prevent the screw from displacing linearly with respect to the first drive element securing member, the screw may be threaded through the second drive element securing member, thereby causing the drive element securing members to displace linearly towards each other on the screw being tightened and to displace linearly away from each other on the screw being loosened.

The robotic surgical instrument as above, wherein the instrument interface element may be linearly displaceable along a displacement axis parallel to a longitudinal axis of a shaft of the instrument.

The robotic surgical instrument as above, wherein the displacement axis may be offset from the longitudinal axis of the shaft.

The robotic surgical instrument may further comprise a linear rail, wherein the instrument interface element may be slidable along the linear rail.

The robotic surgical instrument as above, wherein the holding member may be displaceable linearly between a minimum displacement position and a maximum displacement position.

The robotic surgical instrument as above, wherein a pair of driving elements may be coupled to with the driving element securing member, the driving element securing member being linearly displaceable within the holding member.

The robotic surgical instrument as above, wherein the driving element securing member may be linearly displaceable along a driving element securing member axis which is parallel to the axis along which the holding member is linearly displaceable.

The robotic surgical instrument as above, wherein an alignment mechanism may comprise a screw adjustment mechanism coupled to the holding member and driving element securing member for adjusting the displacement position of the holding member without displacing the driving element securing member.

The robotic surgical instrument as above, wherein the screw adjustment mechanism may comprise a screw threaded into the driving element securing member through a slot in the holding member, the slot being aligned with the driving element securing member axis, the screw being constrained to slide along the slot, thereby permitting the holding member to be displaced relative to the driving element securing member when the screw is loose, and causing the holding member to be held fast with the driving element securing member when the screw is tight.

The robotic surgical instrument as above, wherein the holding member may be a first capstan block that may be configured to be rotatable; the driving element securing member may be a second capstan block that may be configured to be rotatable; and wherein the recess may be a through hole in the first capstan block.

The robotic surgical instrument as above, wherein the first capstan block may comprise: a first half capstan block; and a second half capstan block; wherein the first and second half capstan blocks may be configured to surround the driving element securing member.

The robotic surgical instrument as above, wherein the through hole may be formed of a first through hole portion formed in the first half capstan block and a second through hole portion formed in the second half capstan block.

The robotic surgical instrument as above, wherein the first capstan block may be configured to rotate relative to the second capstan block to tension the at least one driving element.

The robotic surgical instrument as above, wherein the first capstan block and the second capstan block may be configured to rotate in unison to change the offset of the end effector; and wherein there may be at least two driving elements, a first driving element that may be configured to wrap around the first capstan block and a second driving element configured to wrap around the second capstan blocks.

The robotic surgical instrument as above, wherein the through hole in the first capstan block may include at least one tapered side wall.

The robotic surgical instrument as above, wherein each of the first half capstan block and the second half capstan block may include a tapered side wall.

The robotic surgical instrument as above, wherein the second capstan block may include at least one tapered side wall that may be configured to contact the at least one tapered side wall of the first capstan block.

The robotic surgical instrument as above, wherein the at least one tapered side wall of the driving element securing member may be tapered from the direction perpendicular to the direction in which the drive element securing member may be received in the holding member by a taper angle of between 60 degrees and 90 degrees.

The robotic surgical instrument as above, wherein the taper angle may be 70 degrees. The above features may be combined as appropriate, as would be apparent to a skilled person, and may be combined with any of the aspects of the examples described herein.

### BRIEF DESCRIPTION OF THE FIGURES

The present invention will now be described by way of example with reference to the accompanying drawings. In the drawings:

FIG. 1 illustrates a surgical robot performing a surgical procedure;

FIG. 2 illustrates a known surgical instrument;

FIG. 3 illustrates a surgical robotic system;

FIGS. 4A and 4B illustrate a distal end of a surgical instrument;

FIGS. 5A, 5B, 5C, 5D and 5E illustrate a pulley arrangement of the distal end of the surgical instrument of FIGS. 4A and 4B in a variety of configurations;

FIGS. 6A and 6B illustrate two views of a surgical instrument;

FIGS. 7A, 7B and 7C illustrate three views of an instrument interface;

FIG. 8 illustrates an example of a driving element securing member;

FIGS. 9A and 9B illustrate a further example of a driving element securing member with covering members and being received in a holding member according to the present disclosure;

FIGS. 10A, 10B and 10C illustrate a first driving element securing member that uses rotational insertion of a driving element;

FIGS. 11A, 11B and 11C illustrate a second driving element securing member that uses rotational insertion of a driving element;

FIG. 12 illustrates a side view of a driving element securing member being received in a recess of a holding member and illustrates the forces acting on each;

FIG. 13 is a graph illustrating how the reaction force varies with the angle of taper;

FIG. 14 shows a perspective view of a drive assembly interface of a terminal link of a robot arm;

FIG. 15 shows a first cross-sectional view of the drive assembly interface shown in FIG. 14 being connected to an instrument interface of an instrument;

FIG. 16 shows a third cross-sectional view of the drive assembly interface shown in FIG. 14 being connected to the instrument interface of an instrument;

FIG. 17 illustrates a plan view of two capstans that are part of the instrument interface;

FIGS. 18 illustrates a cross-sectional view of the rotational instrument interface element;

FIGS. 19 illustrates a cross-sectional view of the rotational instrument interface element in which the inner wall of the bottom capstan block includes a groove and an outer wall of the top capstan block engages the groove;

FIG. 20 illustrates a cross-sectional view of an alternate rotational instrument interface element;

FIG. 21 illustrates a method of securing a driving element in a driving element securing member;

FIG. 22A is a first view of a third driving element securing member that uses rotational insertion of a driving element;

FIG. 22B is a second view of the third driving element securing member that uses rotational insertion of a driving element.

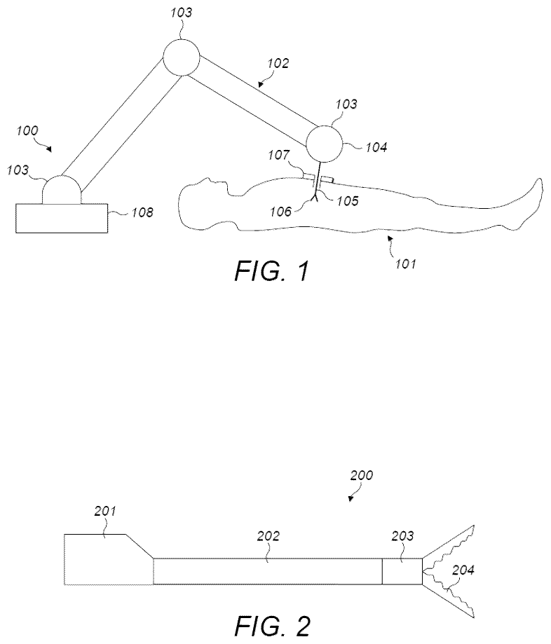

The accompanying drawings illustrate various examples. The skilled person will appreciate that the illustrated element boundaries (e.g., boxes, groups of boxes, or other shapes) in the drawings represent one example of the boundaries. It may be that in some examples, one element may be designed as multiple elements or that multiple elements may be designed as one element. Common reference numerals are used throughout the figures, where appropriate, to indicate similar features.

### DETAILED DESCRIPTION

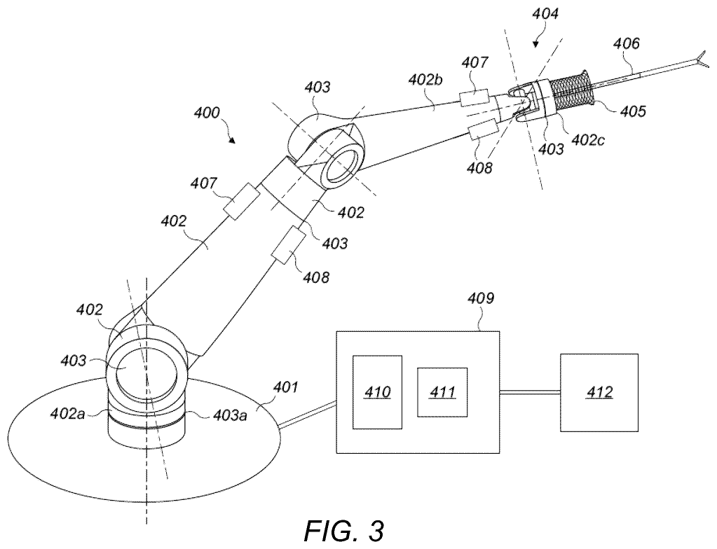

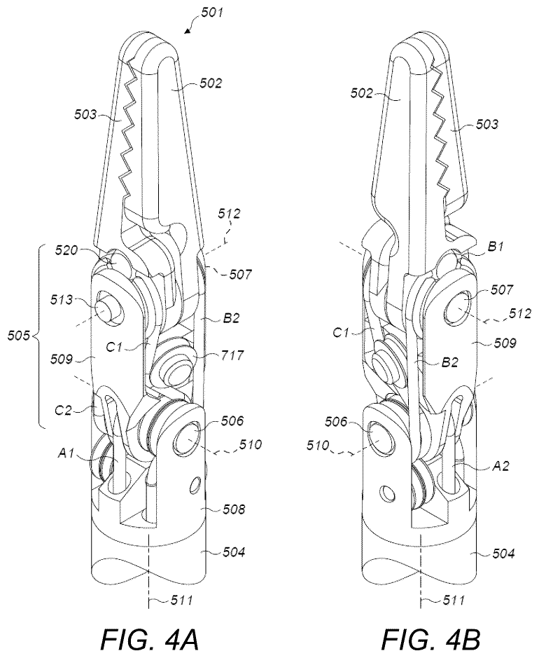

FIG. 3 illustrates a surgical robot having an arm 400 which extends from a base 401. The arm comprises a number of rigid limbs 402. The limbs are coupled by revolute joints 403. The most proximal limb 402a is coupled to the base by joint 403a. It and the other limbs are coupled in series by further ones of the joints 403. FIG. 3 shows a wrist 404 which is made up of four individual revolute joints. The wrist 404 couples one limb (402b) to the most distal limb (402c) of the arm. The most distal limb 402c carries an attachment 405 for a surgical instrument 406. Each joint 403 of the arm has one or more motors 407 which can be operated to cause rotational motion at the respective joint, and one or more position and/or torque sensors 408 which provide information regarding the current configuration and/or load at that joint. Suitably, the motors are arranged proximally of the joints whose motion they drive, so as to improve weight distribution. For clarity, only some of the motors and sensors are shown in FIG. 4. The arm may be generally as described in our co-pending patent application PCT/GB2014/053523.

The arm terminates in an attachment 405 for interfacing with the instrument 406. The instrument 406 may take the form described with respect to FIG. 2. The instrument has a diameter less than 8 mm. For example, the instrument may have a 5 mm diameter. The instrument may have a diameter which is less than 5 mm. The instrument diameter may be the diameter of the shaft. The instrument diameter may be the diameter of the profile of the articulation. The diameter of the profile of the articulation may match or be narrower than the diameter of the shaft. The attachment 405 comprises a drive assembly for driving articulation of the instrument. Movable interface elements of the drive assembly interface mechanically engage corresponding movable interface elements of the instrument interface in order to transfer drive from the robot arm to the instrument. One instrument is exchanged for another several times during a typical operation. Thus, the instrument is attachable and detachable from the robot arm during the operation. Features of the drive assembly interface and the instrument interface aid their alignment when brought into engagement with each other, so as to reduce the accuracy with which they need to be aligned by the user.

The instrument 406 comprises an end effector for performing an operation. The end effector may take any suitable form. To give some examples, the end effector may be smooth jaws, serrated jaws, a gripper, a pair of shears, a needle for suturing, a camera, a laser, a knife, a stapler, a cauteriser, or a suctioner. As described with respect to FIG. 2, the instrument comprises an articulation between the instrument shaft and the end effector. The articulation comprises several joints which permit the end effector to move relative to the shaft of the instrument. The joints in the articulation are actuated by driving elements, such as cables. These driving elements are secured at the other end of the instrument shaft to the interface elements of the instrument interface. Thus, the robot arm transfers drive to the end effector as follows: movement of a drive assembly interface element moves an instrument interface element which moves a driving element which moves a joint of the articulation which moves the end effector.

Controllers for the motors, torque sensors and encoders are distributed with the robot arm. The controllers are connected via a communication bus to control unit 409. The control unit 409 comprises a processor 410 and a memory 411. Memory 411 stores in a non-transient way software that is executable by the processor to control the operation of the motors 407 to cause the arm 400 to operate in the manner described herein. In particular, the software can control the processor 410 to cause the motors (for example via distributed controllers) to drive in dependence on inputs from the sensors 408 and from a surgeon command interface 412. The control unit 409 is coupled to the motors 407 for driving them in accordance with outputs generated by execution of the software. The control unit 409 is coupled to the sensors 408 for receiving sensed input from the sensors, and to the command interface 412 for receiving input from it. The respective couplings may, for example, each be electrical or optical cables, or may be provided by a wireless connection. The command interface 412 comprises one or more input devices whereby a user can request motion of the end effector in a desired way. The input devices could, for example, be manually operable mechanical input devices such as control handles or joysticks, or contactless input devices such as optical gesture sensors. The software stored in memory 411 is configured to respond to those inputs and cause the joints of the arm and instrument to move accordingly, in compliance with a pre-determined control strategy. The control strategy may include safety features which moderate the motion of the arm and instrument in response to command inputs. Thus, in summary, a surgeon at the command interface 412 can control the instrument 406 to move in such a way as to perform a desired surgical procedure. The control unit 409 and/or the command interface 412 may be remote from the arm 400.

FIGS. 4A and 4B illustrate opposing views of the distal end of a surgical instrument. In FIGS. 4A and 4B, the end effector 501 comprises a pair of end effector elements 502, 503, which in FIGS. 4A and 4B are depicted as a pair of opposing serrated jaws. It will be understood that this is for illustrative purposes only. The end effector may take any suitable form, such as those described above. The end effector 501 is connected to the shaft 504 by articulation 505. Articulation 505 comprises joints which permit the end effector 501 to move relative to the shaft 504. A first joint 506 permits the end effector 501 to rotate about a first axis 510. The first axis 510 is transverse to the longitudinal axis of the shaft 511. A second joint 507 permits the first end effector element 502 to rotate about a second axis 512. The second axis 512 is transverse to the first axis 510. A third joint 513 permits the second end effector element 503 to rotate about the second axis 512. The first end effector element 502 and the second end effector element 503 may be independently rotatable about the second axis 512 by the second and third joints. The end effector elements may be rotated in the same direction or different directions by the second and third joints. The first end effector element 502 may be rotated about the second axis, whilst the second end effector element 503 is not rotated about the second axis. The second end effector element 503 may be rotated about the second axis, whilst the first end effector element 502 not rotated about the second axis.

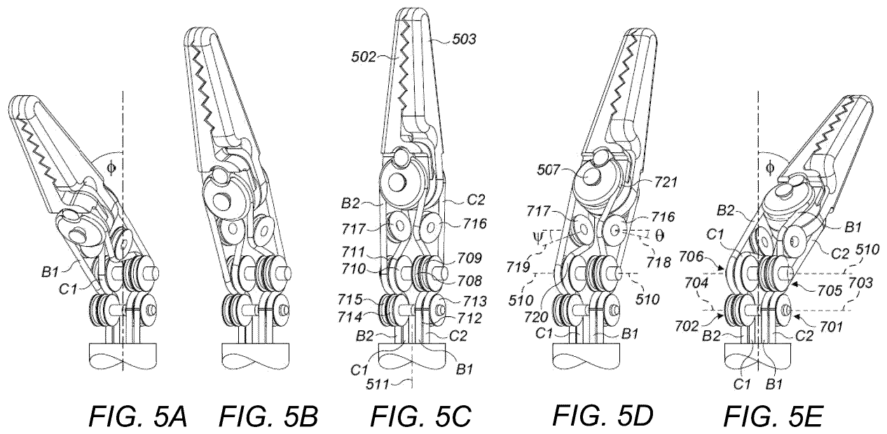

FIGS. 4A and 4B depict a straight configuration of the surgical instrument in which the end effector is aligned with the shaft. In this orientation, the longitudinal axis of the shaft 511 is coincident with the longitudinal axis of the articulation and the longitudinal axis of the end effector. Articulation of the first, second and third joints enables the end effector to take a range of attitudes relative to the shaft. FIG. 5 illustrates some of the configurations of the distal end of the instrument in which articulation about all the first, second and third joints has been driven relative to the straight configuration of FIGS. 4A and 4B.

Returning to FIGS. 4A and 4B, the shaft terminates at its distal end in the first joint 506. The articulation 505 comprises a supporting body 509. At one end, the supporting body 509 is connected to the shaft 504 by the first joint 506. At its other end, the supporting body 509 is connected to the end effector 501 by second joint 507 and third joint 513. Thus, first joint 506 permits the supporting body 509 to rotate relative to the shaft 504 about the first axis 510; and the second joint 507 and third joint 513 permit the end effector elements 502, 503 to rotate relative to the supporting body 509 about the second axis 512.

The joints of the articulation are driven by driving elements. The driving elements are elongate elements which extend from the joints in the articulation through the shaft to the instrument interface. Each driving element can be flexed laterally to its main extent at least in those regions where it engages the internal components of the articulation and instrument interface. In other words, each driving element can be flexed transverse to its longitudinal axis in the specified regions. This flexibility enables the driving elements to wrap around the internal structure of the instrument, such as the joints and pulleys. The driving elements may be wholly flexible transverse to their longitudinal axes. The driving elements are not flexible along their main extents. The driving elements resist compression and tension forces applied along their length. In other words, the driving elements resist compression and tension forces acting in the direction of their longitudinal axes. Thus, the driving elements are able to transfer drive from the instrument interface to the joints. The driving elements may be cables.

Each joint may be driven by a pair of driving elements. Referring to FIGS. 4A and 4B, the first joint 506 is driven by a first pair of driving elements A1,A2. The second joint 507 is driven by a second pair of driving elements 61,62. The third joint is driven by a third pair of driving elements C1,C2. In the example shown in FIGS. 4A and 4B, each joint is driven by its own pair of driving elements. In other words, each joint is driven by a dedicated pair of driving elements. The joints may be independently driven. A pair of driving elements may be constructed as a single piece as shown for the third pair of driving elements in FIGS. 4A and 4B. In this case, the single piece is secured to the joint at one point. For example, the third pair of driving elements C1,C2 comprises a ball feature 520 which is secured to the third joint 513. The ball feature is used by way of example only and other “crimp” types are also possible, for example having different shapes, such as a cylindrical shape. This ensures that when the pair of driving elements is driven, the drive is transferred to motion of the joint about its axis. Alternatively, a pair of driving elements may be constructed as two pieces. In this case, each separate piece is secured to the joint.

FIG. 5 illustrates the distal end of the surgical instrument in five different configurations. Configuration (5C) is the straight configuration previously mentioned, in which the end effector is aligned with the instrument shaft. In configurations (5A), (56), (5D) and (5E), rotation about the first joint has occurred relative to configuration (5C). In configurations (5A), (5B), (5D) and (5E), no rotation about either the second or third joint has occurred relative to configuration (5C). Starting from configuration (5C), the driving element A2 (not shown) is pulled in order to cause the rotation about the first axis 510 leading to the arrangement of configuration (5B). The driving element A2 is further pulled to cause further rotation about the first axis 510 to lead to the arrangement of configuration (5A). Starting from configuration (5C), the driving element A1 (not shown) is pulled in order to cause rotation about the first axis 510 in an opposing direction to that in configurations (5A) and (5B), thereby leading to the arrangement of configuration (5D). The driving element A1 is further pulled to cause further rotation about the first axis 510 to lead to the arrangement of configuration (5E).

Rotation of the end effector 501 about the first axis 510 is bounded by the maximum travel of the first pair of driving elements A1,A2 about the first joint 506. Configuration (5A) shows the end effector 501 at maximum rotation about the first axis 510 in one direction, and configuration (5E) shows the end effector 501 at maximum rotation about the first axis 510 in the opposing direction. The maximum rotation angle relative to the longitudinal axis of the shaft 511 in both configurations is the angle ϕ.

The second and third pairs of driving elements are retained in contact with a first set of pulleys (705 and 706) and a second set of pulleys (701 and 702) for all rotation angles of the end effector relative to the longitudinal axis of the shaft. Thus, regardless of the rotation about the first joint 506, the length of the second pair of driving elements B1,B2 will be maintained the same. Also, regardless of the rotation about the first joint 506, the length of the third pair of driving elements C1,C2 will be maintained the same. Thus, the second set of pulleys enable tension to be retained in the second and third driving elements regardless of how the first joint 506 is driven about the first axis 510. Thus, control of the second and third driving elements is retained regardless of how the first joint 506 is driven about the first axis 510.

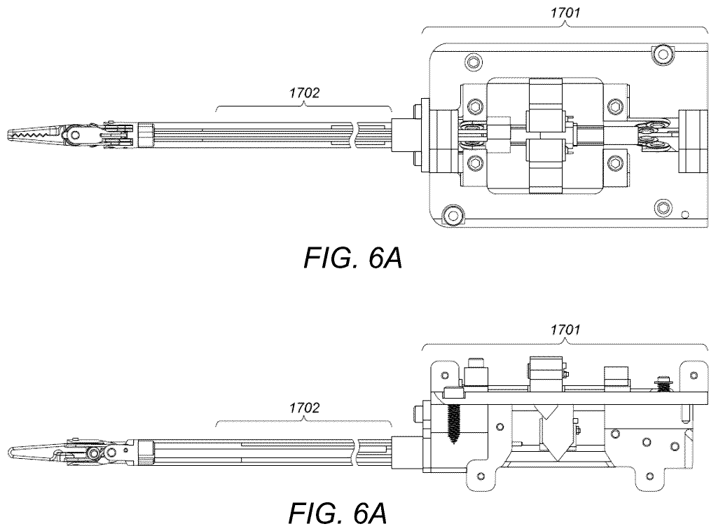

The first, second and third pairs of driving elements extend through the instrument shaft from the distal end of the shaft connected to the articulation to the proximal end of the shaft connected to a drive mechanism of the instrument interface. FIGS. 6A and 6B illustrate two views of the first, second and third pairs of driving elements extending from the described articulation to an exemplary instrument interface 1701.

As can be seen in FIGS. 6A and 6B, the instrument interface may be relatively flat. The instrument interface extends mostly in a central plane viewed head on in FIG. 6A. The instrument shaft 504 may be rigidly attached to the instrument interface 1701. The instrument shaft 504 does not rotate or otherwise move relative to the instrument interface 1701. The second axis 512 about which the end effector elements 502, 503 rotate may be perpendicular to the longitudinal axis 511 of the shaft when the instrument is in the straight configuration (e.g. as shown in FIGS. 4A, 4B and 5C). Thus, in the straight configuration of the instrument, the jaws of the end effector are moveable in the central plane of the instrument interface.

A driving element may be a uniform component having the same shape and size along its length and constructed of the same material along its length. Alternatively, the driving element may be composed of different portions. In one example, the portion of the driving element which engages components of the instrument interface (such as pulleys and interface elements) is flexible. Similarly, the portion of the driving element which engages components of the distal end of the surgical instrument (such as the pulleys and joints in the articulation) is flexible. Between these two flexible portions may be spokes 1702 illustrated in FIGS. 6A and 6B. Thus, each pair of driving elements comprises two spokes and two flexible portions. The spokes are stiffer than the flexible portions. The spokes may be rigid. The spokes may be hollow. The flexible portions may be cables.

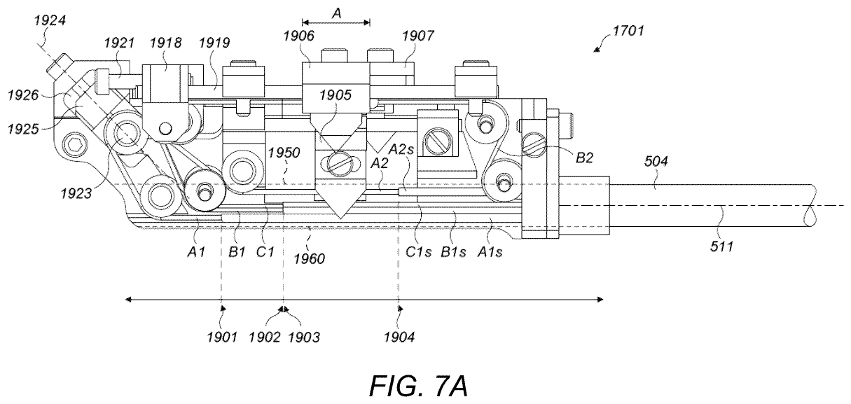

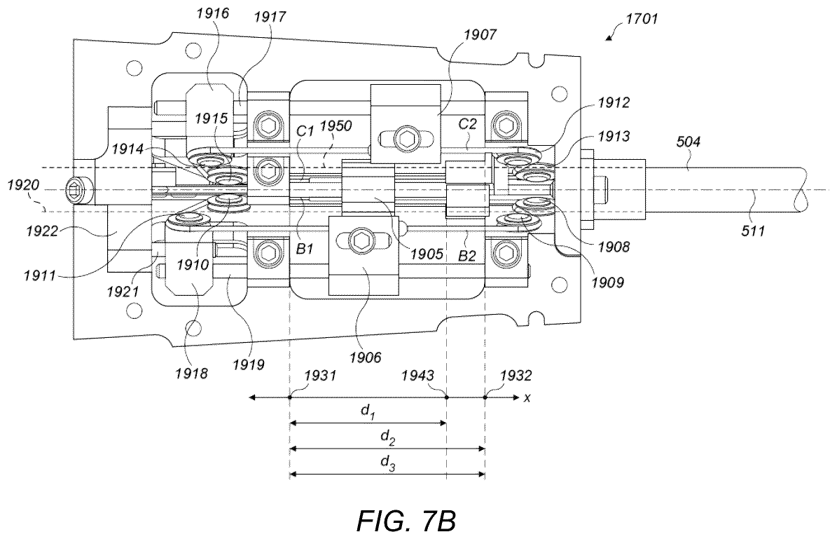

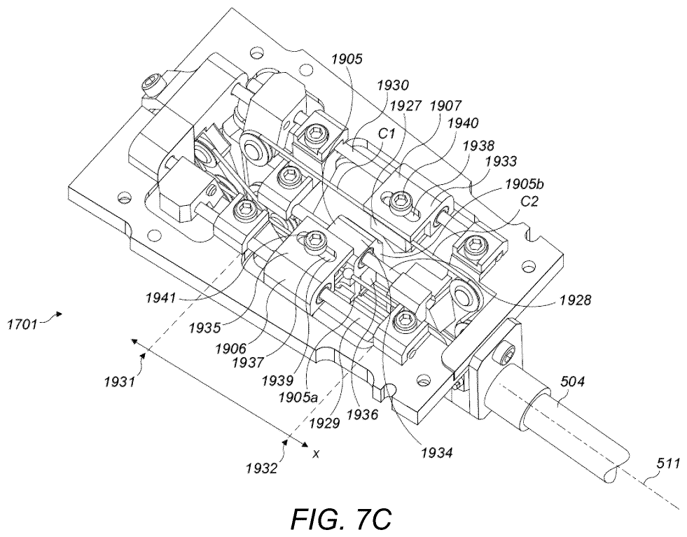

Each driving element engages an instrument interface element in the instrument interface. In the example illustrated in FIGS. 7A, 7B and 7C, each driving element engages its own respective instrument interface element. In other words, there may be a one-to-one relationship between the instrument interface elements and the driving elements. A single instrument interface element drives a single driving element. Each driving element is driven independently by a single instrument interface. In alternative arrangements, there may be a compound driving motion in which more than one instrument interface element drives a single driving element, a single instrument interface element drives more than one driving element, or a plurality of instrument interface elements collectively drive a plurality of driving elements.

FIGS. 7A, 7B and 7C illustrate a first embodiment in which a first instrument interface element 1905 engages the first pair of driving elements A1, A2. A second instrument interface element 1906 engages the second pair of driving elements B1, B2. A third instrument interface element 1907 engages the third pair of driving elements C1, C2. Each driving element is secured to its associated instrument interface element. In other words, each driving element is coupled to its associated instrument interface element.

The instrument interface 1701 has a significantly larger profile than the instrument shaft 504. Typically, the instrument shaft has a circular cross-section having a diameter of less than or the same as 5 mm, whereas a corresponding cross-section through the instrument interface may be larger than this. The instrument interface comprises an internal portion and an external portion. The internal portion is bounded by the dotted line 1950. The external portion is the remainder of the instrument interface which is not in the internal portion. The internal portion is within the projected profile of the shaft. In other words, the internal portion is the part of the instrument interface that would have been encompassed had the profile of the shaft continued within the instrument interface. The external portion is outside of the projected profile of the shaft. In the example illustrated, the shaft has a constant circular cross-section, and hence the internal portion is a cylinder having the same circular cross-section as the shaft, and having the same longitudinal axis 511 as the shaft. In other words, the internal portion is an extrapolation of the constant cross-section of the shaft in the instrument interface. The internal portion 1950 is shown from the side in FIG. 7A and from the top in FIG. 7B.

Instrument interface element 1905 engages a first pair of driving elements A1, A2. Instrument interface element 1906 engages a second pair of driving elements B1, B2. Instrument interface element 1907 engages a third pair of driving elements C1, C2.

A pulley arrangement is used to shift the driving elements over to engage with the instrument interface elements which are in the external portion. Each pair of driving elements engages a first pair of pulleys to shift it over from the proximal end of the shaft 504 to its respective instrument interface element, and a second pair of pulleys to shift it back from alignment with the instrument interface element to alignment with the shaft 504.

In the arrangement shown, the second pair of driving elements B1, B2 emerges from the proximal end of the shaft in a direction aligned with the shaft. The second pair of driving elements B1, B2 is then constrained to move around pulley pair 1908 and 1909 to shift it from where it emerges from the shaft 504 to engagement with the second instrument interface element 1906. The second pair of driving elements B1, B2 emerges from the pulley pair 1908 and 1909 in a direction parallel to and offset from the direction that the second pair of driving elements B1, B2 emerges from the proximal end of the shaft. The second pair of driving elements B1, B2 is constrained to move around pulley pair 1910 and 1911 to shift it from alignment with the second instrument interface element 1906 to alignment with the shaft 504.

In the arrangement shown, the third pair of driving elements C1, C2 emerges from the proximal end of the shaft in a direction aligned with the shaft. The third pair of driving elements C1, C2 is then constrained to move around pulley pair 1912 and 1913 to shift it from where it emerges from the shaft 504 to engagement with the third instrument interface element 1907. The third pair of driving elements C1, C2 emerges from the pulley pair 1912 and 1913 in a direction parallel to and offset from the direction that the third pair of driving elements C1, C2 emerges from the proximal end of the shaft. The third pair of driving elements C1, C2 is constrained to move around pulley pair 1914 and 1915 to shift it from alignment with the third instrument interface element 1907 to alignment with the shaft 504.

In the arrangement shown in FIGS. 7A, 7B and 7C, the first pair of driving elements A1, A2 engage with the first instrument interface element 1905 which is within the internal portion. The first pair of driving elements A1, A2 drive rotation of the articulation, and hence the end effector, about the first axis 510 (see FIG. 4A).

Each instrument interface element is displaceable within the instrument interface. Since each instrument interface element is secured to a corresponding pair of driving elements, a displacement of the instrument interface element is transferred to a displacement of the pair of driving elements. Suitably, each instrument interface element is displaceable along the same line as the line of the pair of driving elements that it is secured to. Each instrument interface element engages with a corresponding drive assembly interface element of the robot arm. Thus, displacement of the instrument interface element is driven by the robot arm. In this way, the robot arm drives the pairs of driving elements.

Each pair of driving elements engages with an instrument interface element in the instrument interface. One or more of the driving elements may engage with a tensioning mechanism and an alignment mechanism. When manufacturing the instrument, the tensioning mechanism is used to achieve a desired tension in the pair of driving elements. The alignment mechanism is used to set the instrument interface elements to a predetermined alignment position when the end effector has a predetermined configuration. Each instrument interface element has a displacement range over which it is displaceable. The predetermined alignment position may be the midpoint of the displacement range for each instrument interface element. The predetermined configuration of the end effector may be the straight configuration, in which the end effector elements are closed together (for example the jaws are closed), and the longitudinal axis of the articulation and the longitudinal axis of the end effector are aligned with the longitudinal axis of the shaft 511. By setting the instrument interface elements to a predetermined alignment position when the end effector has a predetermined configuration, when changing instruments during an operation, the time taken to set up the new instrument ready for use is reduced. In practice, when an instrument is removed from the robot arm, the robot arm assembly is configured to go to an arrangement in which it is ready to receive the instrument interface elements in the predetermined alignment position. For example, the robot arm assembly interface elements go to a default position in which they are arranged to receive each of the instrument interface elements at the midpoint of their displacement range. Then, the instrument is manually put in the predetermined configuration and then slotted into the robot arm. For example, the technician moves the articulation and end effector into the straight configuration and then slots the instrument into the robot arm. Because it is known that the instrument interface elements have the predetermined alignment position when the instrument is in the predetermined configuration, the instrument interface elements engage directly with the robot arm assembly interface elements. The control system does not need to perform an additional calibration or software setup procedure in order to map the position and orientation of the end effector, because it is known that the end effector is in the predetermined configuration.

The following describes tensioning and alignment mechanisms which are independent of each other. By isolating the tensioning mechanism from the alignment mechanism the process by which the desired tension and desired alignment are achieved is simplified. Thus, the time taken to achieve the desired tension and desired alignment during manufacture is reduced.

FIGS. 7A, 7B and 7C illustrate a tensioning mechanism utilising pulleys. Each pair of driving elements is independently tensioned. Each pair of driving elements is constrained to move around a pulley which is displaceable. FIGS. 7A, 7B and 7C depict two different exemplary pulley arrangements for tensioning the pairs of driving elements. In both examples, the pulley is linearly displaceable.

Referring firstly to the tensioning mechanism shown for the pairs of driving elements B1,B2 and C1,C2. Taking pair of driving elements B1,B2 first, pulley 1911 is used to tension B1,B2. Pulley 1911 is linearly displaceable along a displacement axis 1920 which is parallel to the longitudinal axis 511 of the shaft. The displacement axis 1920 is offset from the longitudinal axis 511 of the shaft. The tensioning pulley 1911 is mounted to a block 1918 which is slidable along a rail 1919. Sliding the block 1918 along the rail 1919 causes the pulley 1911 to displace along the displacement axis 1920. When the block 1918 is moved away from the shaft, the tension of the second pair of driving elements B1,B2 increases. When the block 1918 is moved towards the shaft, the tension of the second pair of driving elements B1,B2 decreases. Any suitable mechanism may be used to move the block. For example, a screw adjustment mechanism may be used. FIGS. 7A, 7B and 7C show a screw adjustment mechanism in which screw 1921 is threaded into block 1918. This is most clearly seen on FIG. 7A. The screw 1921 is constrained by portion 1922 of the instrument interface such that it is able to rotate but not able to be displaced linearly. Thus, when the screw is rotated, the screw thread engages with the thread inside the block 1918 causing the block and hence the pulley 1911 to displace linearly. When the screw 1921 is tightened, the pulley 1911 moves in one linear direction. When the screw 1921 is loosened, the pulley 1911 moves in the opposing linear direction. The tensioning mechanism for driving elements C1,C2 depicted in FIGS. 7A, 7B and 7C works in a corresponding manner to that described with relation to driving elements 81,82.

Referring now to the tensioning mechanism shown for the first pair of driving elements A1,A2 in FIG. 7A. Pulley 1923 is used to tension A1, A2. Pulley 1923 is linearly displaceable along a displacement axis 1924. Displacement axis 1924 is at an angle to the longitudinal axis 511 of the shaft. Suitably, the displacement axis 1924 may be at a 45° angle to the longitudinal axis 511 of the shaft. The tensioning pulley 1923 is mounted to a block 1925 which is captive in a socket 1926 of the instrument interface. The block 1925 and tensioning pulley 1923 are able to slide through the socket 1926. Sliding the block 1925 through the socket 1926 causes the pulley to displace along the displacement axis 1924. When the block 1925 is slid further into the socket, the tension of the first pair of driving elements A1,A2 increases. When the block 1925 is slid out of the socket, the tension of the first pair of driving elements A1,A2 decreases. Any suitable mechanism may be used to move the block 1925. For example, a screw adjustment mechanism as described above with respect to block 1918 may be used. Since this tensioning mechanism moves tensioning pulley 1923 at 45° to the longitudinal axis of the shaft 511.

Although FIGS. 7A, 7B and 7C show the first pair of driving elements using the angled tensioning mechanism, and the second and third pairs of driving elements using the linear tensioning mechanism, any pair of driving elements may be tensioned using any suitable mechanism as long as that mechanism packages into the instrument interface.

Referring to FIG. 7C, each instrument interface element 1905, 1906 and 1907 may be linearly displaceable parallel to the longitudinal axis of the shaft 511. The instrument interface element may be slidable along a linear rail. For example, first instrument interface element 1905 is slidable along rail 1928, second instrument interface element 1906 is slidable along rail 1929, and third instrument interface element 1907 is slidable along rail 1930. Each instrument interface element can be displaced over a displacement range between a minimum displacement position and a maximum displacement position. For example, the minimum and maximum displacement positions may be determined by the ends of the rail along which the instrument interface element slides in the longitudinal direction x of the shaft. The minimum and maximum displacement positions are labelled 1931 and 1932 on FIGS. 7b and 7c for the second and third instrument interface elements 1906 and 1907. The minimum and maximum displacement positions are labelled 1931 and 1943 on FIG. 7b for the first instrument interface element 1905. The first instrument interface element is linearly displaceable through a maximum distance d₁. The second instrument interface element is linearly displaceable through a maximum distance d₂. The third instrument interface element is linearly displaceable through a maximum distance d₃. Suitably d₁<d₂ and d₁<d₃ . Suitably, d₂=d₃.

Suitably, in the straight configuration of the instrument in which the end effector is aligned with the shaft, the first, second and third instrument interface elements 1905, 1906 and 1907 are all located in the same plane perpendicular to the longitudinal axis of the shaft.

Suitably, in the first embodiment each instrument interface element comprises a holding member (body) 1933, 1934, 1935 and a driving element securing member (lug) 1927, 1936, 1937. The body 1933, 1934, 1935 is linearly displaceable between the minimum displacement position and the maximum displacement position of the instrument interface element. The one or more driving elements which engage the instrument interface element is secured to the lug of the instrument interface element. The lug may be linearly displaceable within the body parallel to the direction along which the body is displaceable. Suitably, the lug is linearly displaceable along the longitudinal direction x of the shaft parallel to the longitudinal axis 511 of the shaft. The alignment mechanism adjusts the displacement position of the body without displacing the lug. For example, the alignment mechanism may comprise a screw adjustment mechanism coupled to the body and lug which enables the body to move without moving the lug. FIG. 7c depicts one such adjustment mechanism using a screw. In this example, the body 1933, 1935 comprises a slot 1938, 1939 aligned with the direction along which the body is displaceable. In other embodiments a through hole may be used instead of the slot. The slot and through hole may be thought of as a first opening. A screw 1940, 1941 or other driving element securing member may be threaded into the lug through the slot 1938, 1939. The screw 1940, 1941 is constrained to slide along the slot. For example, the screw head may be too large to pass through the slot or through hole and the screw body a loose fit through the slot. Thus, when the screw is loose, the body is displaceable relative to the lug along the length of the slot. When the screw is tight, the body is held fast with the lug. Thus, the relative position of the body and the lug can be adjusted by the width of the slot.

The articulation may be driven by at least one driving element. Furthermore, in relation to the instrument interface in order to adjust the tension of the one or more driving elements as well as perform instrument zeroing the linear position of the holding member and the driving element securing member can be changed. In order to achieve this, the one or more driving elements must be securely retained by the driving element securing member and in turn this driving element securing member is securely retained by the holding member. The driving elements may be removably retained by the driving element securing member such that they can be replaced when required. Similarly, the driving element securing member may be removably retained in the holding member by means of a fixing member which may be a screw, or similar, described above, as well as frictional forces.

The driving element securing member will now be described in detail, in particular how the driving elements are secured by the driving element securing member. For the purpose of this description the driving element securing member will be referred to as the cable end block and the driving elements will be referred to as cables and may have crimps on the terminal ends (end portions) of the driving elements. These crimps may be the portion of the driving elements that are retained by the cable end block. The crimp on the end of a driving element has a larger cross-sectional area than the rest of the driving element (where the “rest of the driving element” may be referred to herein as the “body portion of the driving element”). The driving element securing member may have a substantially cuboid shape, preferably a rectangular cuboid, and comprise at least one tapered side wall that is configured to engage with at least one wall (e.g. a tapered wall) of the holding member. The at least one tapered side wall of the driving element securing member is configured to be coupled with the at least one driving element. The driving element securing member also includes a through hole, either side of which the tapered side walls may be arranged when there is more than one tapered side wall.

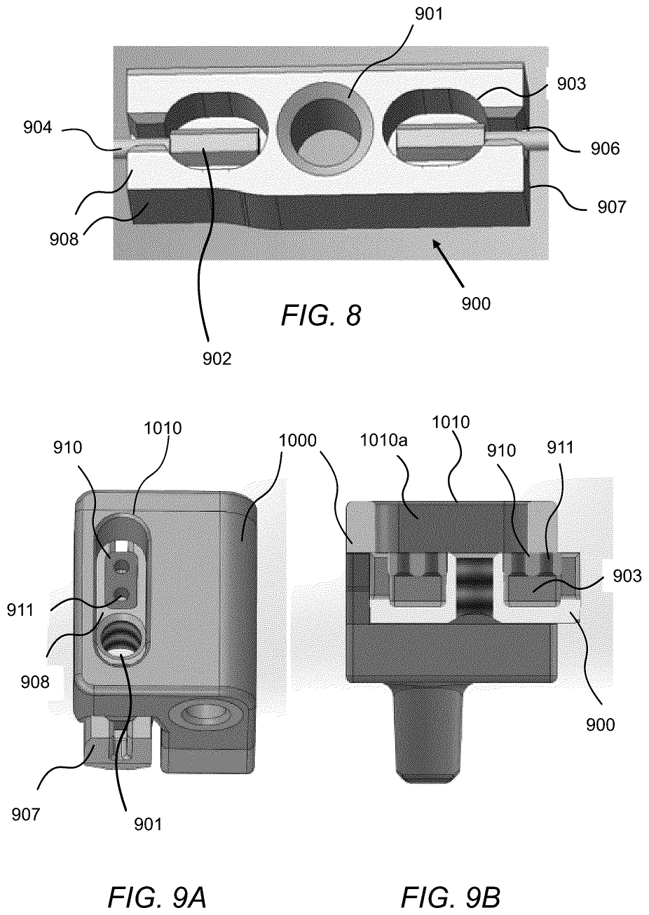

In the linear embodiment of the instrument interface, the cable end blocks 900 are configured to retain the cable crimps 902 in the manner shown in FIG. 8. In general cable crimps 902 are applied to the ends of cables 904 to allow the cables 904 to be retained by a channel 906 that has a cross section that is wider than the cable 904 (the “body portion” of the driving element) but narrower than the cable crimp 902 (the “end portion” of the driving element). As shown in FIG. 8, cable end blocks 900 may include one or more openings defining a cavity 903 configured to accommodate the cable crimps 902. The cavity may be partially enclosed and comprise openings on the side faces 908 of the substantially rectangular cable end block 900. The cables 904 to which the cable crimps 902 are connected are configured to exit the cavity 903 in the cable end block 900 through a hole 906 in the end face 907 of the cable end block 900. The plane in which the opening in the end face 907 is formed is perpendicular to the one or more planes in which the one or more openings are formed in the one or more side faces 908 of the cable end block 900. Driving elements 904 and cable crimps 902 are placed under tension once in place in the cavity 903 of the driving element securing member 900. The cable crimp 902 of a driving element 904 maintains the driving element 904 in an orientation such that the driving element 904 does not undergo bending. In addition, the driving elements 904 being under tensions further ensures that the load exerted on the crimps 902 that connect to the driving elements 904, and are held by the driving element securing member 900, are loaded axially. That is the load applied to the crimps 902 is in the longitudinal direction of the shaft and the instrument interface.

However, in this configuration one cause of failure in the retaining of the cables 904 (driving elements) by the cable end block 900 (driving element securing member) may be that as tension applied to the cable 904 breaches a threshold or as the end effector moves, the cables 904 become detached/come out of the cable end blocks. This separates the cable 904 and the cable end blocks 900, e.g., the cable crimps 902 detach from the cable end blocks 900. In other words, the driving elements 904 are no longer retained by the driving element securing member 900 leading to loss of control of the driving element 904 and inability to manoeuvre the end effector of the instrument.

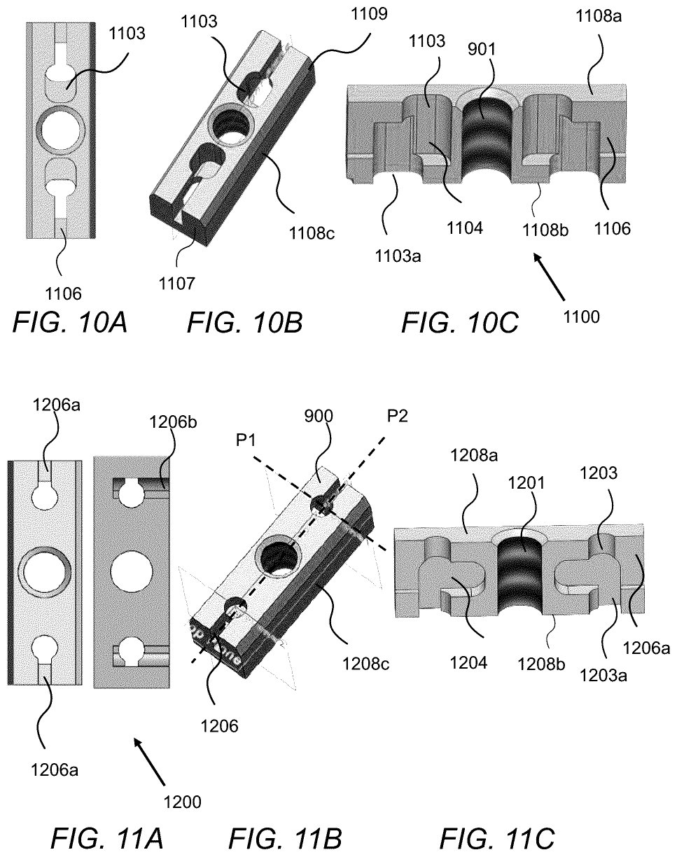

This may be solved in two ways, each of which will now be described in relation to FIGS. 9A, 9B and 10.

FIGS. 9A and 9B depict a driving element securing member 900 comprising a groove lid 910 (which may be referred to as a “covering member”). The purpose of the groove lid 910 is to enclose the cable crimp 902 further retaining it in the cavity 903 of the driving element securing member 900 and thus preventing the crimp 902 from detaching (pulling out) of the driving element securing member 900. In this solution this is achieved in the following way. The driving element securing member 900 has a substantially rectangular cross section similar to that shown in FIG. 8, however an opening to the cavity 903 is formed in only one side face 908 of the driving element securing member 900. The side face 908 that the opening is formed in may be any face that is not the end face 907 of the driving element securing member 900. For example, the opening may be formed in the side face 908 that opens to the slot/through hole 1010 in the holding member 1000 as shown in FIG. 9A, this side face 908 may also be considered the “top” face. This opening in the side face is larger than the crimp 902 in order to facilitate the initial crimp placement as shown in FIG. 8. Once the crimp 902 is placed in the cavity 903, the opening of the cavity 903 is covered with a covering member (groove lid) 910 configured to retain the crimp 902 in the cavity 903 by preventing the crimp 902 from dislodging from/popping out of the cavity 903 through the opening.

The covering member 910 may be provided with one or more through holes 911 that are arranged to connect the cavity 903a (the void that the opening forms) of the opening 903 with the cavity 1010a of the slot 1010. In other words, the one or more through holes 911 are arranged to communicate with the cavity 903 formed by the opening and the outside atmosphere/the outside of the driving element securing member 900. The covering member 910 may be formed of a solid or deformable material and may be press fit into the opening 903 to prevent the crimp 902 from detaching from the drive element securing member 900. The through holes 911 in the covering member 910 prevent the build-up of water or other liquid in the cavity 903, which could lead to the crimp 902 being forced out of the cavity 903. The through holes 911 thus allow liquid to drain from the cavity 903 while also reducing the stiffness of the covering member 910 itself. In some cases, the covering member may have two or more through holes 911 formed in it. Furthermore, the driving element securing member 900 may have one or more, in particular two, openings as described above formed in it. The more than one opening may be arranged either side of a first opening 901 configured to receive the fixing member 801 for fixing the driving element securing member to a holding member of an instrument interface element as described below. In the above-described configuration shown in FIG. 8 the crimp 902 is horizontally inserted into the driving element securing member 900 by pushing it through the opening into the cavity 903 from the direction in which the cavity 903 formed by the opening in the driving element securing member 900 extends. “Horizontally” in this configuration means such that the driving element 904 is positioned to extend perpendicular to the direction in which the cavity 903 formed by the opening in the side face in the driving element securing member 900 extends from that side face. As such, it is necessary for the opening to be larger than the length of the crimp.

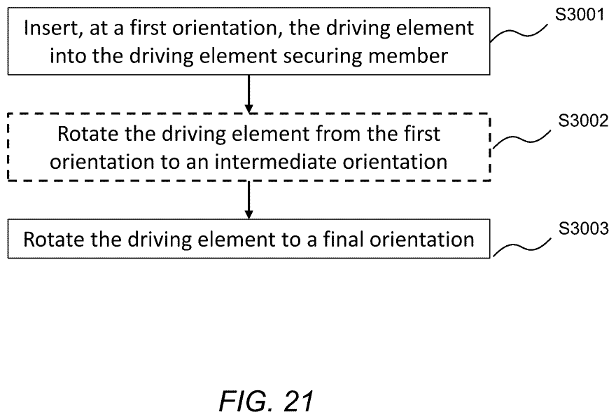

In an alternate configuration of the driving element securing member 1100 shown in FIG. 10 the crimp 902 (end portion) may be rotationally inserted into the driving element securing member 1100 in order to retain the crimp 902. In these configurations the crimp 902 is inserted (e.g. in step S3001 of the flow chart shown in FIG. 21, which is described below) into a cavity 1104 in the driving element securing member 1100 at an angle through an opening 1103, and thus the opening 1103 does not need to be larger than the length of the crimp 902 (end portion of the driving element). The orientation of the driving element when it is being inserted may be thought of as a first orientation. The crimp 902 enters the opening 1103 formed in the driving element securing member 1100 at an angle, e.g., in an orientation that is not parallel(e.g., approximately perpendicular) to a longitudinal direction of the driving element securing member and to the final orientation seen in FIG. 8, and is then rotated (e.g. in step S3003 of the flow chart shown in FIG. 21) to its final position (final orientation) such that it is oriented in the longitudinal direction of the driving element securing member as shown in FIG. 8 within the cavity 1103. As can be seen in FIGS. 10A, 108 and 10C the opening 1103 formed in the side face (top) 1108a of the driving element securing member 1100 in FIG. 10C and the opening 1103a formed in the side face (bottom) 1108b of the driving element securing member 1100 in FIG. 10C are smaller than the openings to the cavity 903 seen in FIGS. 8, 9A and 9B. In this configuration a first opening 1103 is formed in one side face 1108a of the driving element securing member 1100 and the cavity 1104 extends partially but not completely from the opening 1103 through the body of the driving element securing member 1100. A further opening 1103a that is offset from the first opening 1103 in the longitudinal direction (e.g. a horizontal direction in FIG. 10C or a vertical direction in FIG. 10A) of the driving element securing member body 1100 is formed in the side face 1108b opposing the side face 1108a in which the first opening 1103 is formed as seen in FIGS. 10A, 108 and 10C. The cavity 1104 extends partially, but not completely from the opening 1103a through the body of the driving element securing member 1100 in the same way as the first opening 1103. The two openings 1103, 1103a overlap each other in the longitudinal direction of the driving element securing member such that the cavity 1104 connects the first opening 1103 and the further opening 1103a. The length of the cavity 1104 in the longitudinal direction may be larger than the length of the crimp 902 in that direction when the crimp 902 is retained within the cavity 1104. This allows the crimp 902, which may enter the first opening 1103 approximately vertically, to pivot/rotate through approximately 90 degrees into a position in which the crimp 902 is arranged in the longitudinal direction of the driving element securing member 1100 as shown in FIG. 8. The driving element securing member 1100 also includes a channel 1106 that communicates with the cavity 1104 formed in the body of the driving element securing member 1100. This channel 1106 opens to one of the side faces 1108a of the driving element securing member and an end face 1107, the plane of which is substantially perpendicular to all of the side faces 1108a, 1108b, 1108c. The channel 1106 allows the cable 904, e.g., the body portion of the driving element, that is connected to the crimp 902 to exit the driving element securing member 1100. The cross section/width of the channel 1106 is smaller than the cross section/width of the crimp 902 and thus the crimp 902 cannot pass through the channel 1106 and exit the driving element securing member 1100. A similar channel 906 is shown in FIG. 8 that allows the driving element 904 to exit the driving element securing member 900.

There are various other opening geometries that allow for rotational insertion of the crimp 902 as shown in FIGS. 10A, 108, 10C, 11A, 11B and 11C of the present application. The specific geometry will dictate the steps that are required in order to insert the crimp 902 into the driving element securing member 1100, 1200. For example, the driving element securing member 1100 shown in FIGS. 10A, 108 and 10C requires only one rotational step, upon first insertion, in order to position the crimp 902 securely orientated in the longitudinal direction of the driving element securing member 1100. Conversely, the geometry of the driving element securing member 1200 shown in FIGS. 11A, 118 and 11C requires more than one step to insert and remove the crimp 902 from the driving element securing member 1200. The left half of FIG. 11A illustrates the top of the driving element securing member 1200 and the right half of FIG. 11A illustrates the bottom of the driving element securing member 1200. In the geometry of the driving element securing member 1200 of FIGS. 11A, 11B and 11C, the body of the driving element securing member 1200 is again a substantially rectangular block, with a first opening 1201 (through hole) formed in through the body and two openings 1203 and 1203a opening to two of the side faces 1208a, 1208b of the driving element securing member 1200. A first opening 1203 is formed in a first side surface 1208a of the driving element securing member 1200. This first opening 1203 connects to a cavity 1204 which extends into the body and is in communication with a first channel 1206a that opens to an end surface of the driving element securing member 1200. A further opening 1203a is formed in the side face 1208b that opposes the first side face 1208a. The further opening 1203a communicates with a further channel 1206b in the body that is perpendicular to the first channel 1206b, and opens to a side face 1208c that is perpendicular to the side faces 1208a, 1208b that the first and further openings 1203, 1203a are formed in. The cavity 1204 connects the first opening 1203 and the further (second) opening 1203a inside the body of the driving element securing member 1200. The first opening 1203 formed in the first side face 1208a and the further opening 1203a formed in the second side face 1208b do not have to be offset from each other in the longitudinal direction of the driving element securing member 1200 but may be. In this configuration each opening 1203, 1203a may be circular hole.

With this driving element securing member 1200 geometry, in order to retain the driving element (e.g., crimp and cable), the crimp 902 (end portion of the driving element) is vertically inserted into the first opening 1203 (in the orientation shown in FIG. 11C) until the crimp 902 passes through the driving element securing member 1200 and partially out of the further opening 1203a. At this point the driving element 904 will be within the cavity 1204. The crimp 902 is then rotated to horizontally extend the in the direction perpendicular to the longitudinal direction of the driving element securing member 1200. This is along a horizontal direction of plane 1 P1 shown in FIG. 11B. The crimp is then rotated within the cavity 1204 again to sit along a plane P2 that is in the direction of the longitudinal axis of the driving element securing member 1200 as shown in FIG. 11B. The crimp is then straightened to align the cable length to pass through the first channel 1206a.

In some cases, the length of one or more of the openings may be smaller than the length of the cavity in the longitudinal direction of the driving element securing member.

In a further example configuration, only one of the two openings described above may have a length in the longitudinal direction of the driving element securing member 1200 that is less than the length of the crimp 902 (end portion of the driving element) in the longitudinal direction of the driving element, while the opening on the opposite side face may have an opening of any length. In other words, in some non-limiting cases, in relation to FIGS. 10A, 108, 10C, 11A, 11B, and 11C, while the openings 1103 and 1203 may remain as described above, openings 1103a and 1203a may have a length that is not less than that of the crimp 902. In this configuration the two openings need not be offset from each other along the longitudinal direction of the driving element securing member 1200. The second opening of this configuration, the length of which is not less than the length of the crimp 902 and/or the cavity 1104, 1204 may therefore be rectangular or other shape that is sufficiently wide and long enough such that the crimp may fit through it without needing to be twisted. Therefore, in such a configuration, the driving element may be inserted (threaded) vertically into the first opening 1103, 1203 on a first side face 1108a, 1208a of the driving element securing member 1200 and then exit the driving element securing member 1200 at a second side surface/face 1108b, 1208b in which the second opening 1103a, 1203a is formed.

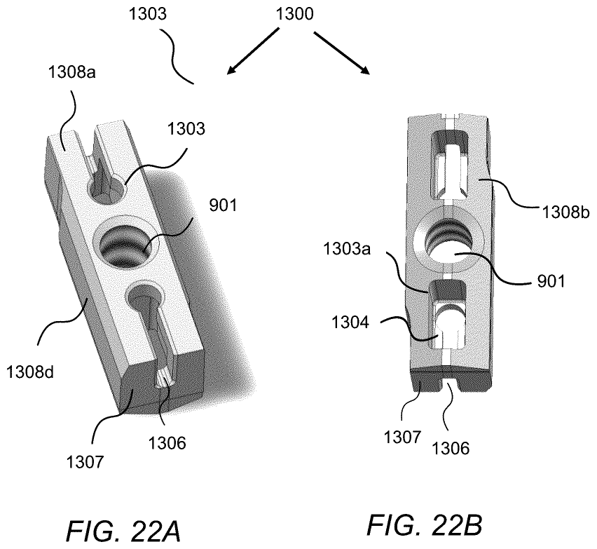

In a further example configuration of the driving element securing member 1100, 1200 shown in FIGS. 10 and 11, an alternate configuration of driving element securing member 1300 is shown in FIG. 22 in which the crimp 902 (end portion) may be rotationally inserted into the driving element securing member 1300 in order to retain the crimp 902. In these configurations the crimp 902 is inserted (e.g., in step S3001 of the flow chart shown in FIG. 21, which is described below) into a cavity 1304 in the driving element securing member 1300 at an angle through an opening 1303, and thus the opening 1303 does not need to be larger in the longitudinal direction of the driving element securing member 1300 than the length of the crimp 902 (end portion of the driving element) along the longitudinal direction of the driving element. The orientation of the driving element when it is being inserted may be thought of as a first orientation. The crimp 902 enters the opening 1303 formed in the driving element securing member 1300 at an angle (e.g., approximately perpendicular), e.g., in an orientation that is not parallel to a longitudinal direction of the driving element securing member 1300 and to the final orientation seen in FIG. 8, and is then rotated (e.g. in step S3003 of the flow chart shown in FIG. 21) to its final position (final orientation) such that it is oriented in the longitudinal direction of the driving element securing member as shown in FIG. 8 within the cavity 1303. As can be seen in FIGS. 22A and 22B the opening 1303 formed in the side face (top) 1308a of the driving element securing member 1300 in FIG. 22A is smaller than the openings to the cavity 903 seen in FIGS. 8, 9A and 9B. In other words, the opening 1303 has a length and in the longitudinal direction of the driving element securing member 1300 that is shorter than the length of the crimp 902 of the driving element. However, unlike opening 1300, the opening 1303a formed in the side face (bottom) 1308b of the driving element securing member 1300 in FIG. 22B that opposes the opening 1303 may not be smaller than the openings to the cavity 903 seen in FIGS. 8, 9A and 9B. In other words, the opening 1303a is not shorter in a longitudinal direction of the driving element securing member 1300 than the crimp 902 of the driving element. In this configuration a first opening 1303 is formed in one side face 1308a of the driving element securing member 1300 and the cavity 1304 extends partially but not completely from the opening 1303 through the body of the driving element securing member 1300. The further opening 1303a in this configuration or the configuration of FIGS. 10 and 11 may not offset from the first opening 1303 in the longitudinal direction (e.g., a vertical direction in FIG. 22B) of the driving element securing member body 1300 is formed in the side face 1308b opposing the side face 1308a in which the first opening 1303 is formed as seen in FIGS. 22A and 22B. The cavity 1304 extends partially, but not completely from the opening 1303a through the body of the driving element securing member 1300 in the same way as the first opening 1303. The two openings 1303, 1303a overlap each other in the longitudinal direction of the driving element securing member such that the cavity 1304 connects the first opening 1303 and the further opening 1103a. The length of the cavity 1304 in the longitudinal direction may be larger than the length of the crimp 902 in that direction when the crimp 902 is retained within the cavity 1104. This allows the crimp 902, which may enter the first opening 1303 approximately vertically, to pivot/rotate through approximately 90 degrees into a position in which the crimp 902 is arranged in the longitudinal direction of the driving element securing member 1300 as shown in FIG. 8. This rotation may also be performed once the driving element has entered the second opening 1303a which has a length that is not smaller than the length of crimp 902 in the longitudinal direction of the driving element securing member 1300. The driving element securing member 1300 also includes a channel 1306 that communicates with the cavity 1304 formed in the body of the driving element securing member 1300. This channel 1306 opens to one of the side faces 1308a of the driving element securing member and an end face 1307, the plane of which is substantially perpendicular to all of the side faces 1308a, 1308b, 1308c. The channel 1306 allows the cable 904, e.g., the body portion of the driving element, that is connected to the crimp 902 to exit the driving element securing member 1300. The cross section/width of the channel 1306 is smaller than the cross section/width of the crimp 902 and thus the crimp 902 cannot pass through the channel 1306 and exit the driving element securing member 1300. A similar channel 906 is shown in FIG. 8 that allows the driving element 904 to exit the driving element securing member 900. The channel is open to the side face 1308a (i.e. the side face on which the opening 1303 has a length that is smaller than the length of the crimp 902 in the longitudinal direction of the driving element securing member 1300), but the channel 1306 is not open to the side face 1308b (i.e. the side face on which the opening 1303a has a length that is not smaller than the length of the crimp 902 in the longitudinal direction of the driving element securing member 1300). This ensures that, although the opening 1303a has a length that is not smaller than the length of the crimp 902 in the longitudinal direction of the driving element securing member 1300, the driving element can be secured in the driving element securing member 1300.

The number of steps required to attached and detach the driving element 902 from the driving element securing member 1100, 1200, 1300 may vary depending on the specific geometry of the driving element securing member 1100, 1200, 1300. However, it is noted that in the above configurations the driving element 902 must be rotated in at least one plane or along one axis in order for the driving element 902 to be attached or detached, which makes accidental detachment of the driving element from the driving element securing member much less likely than in the case shown in FIG. 8.

The method, described above in relation to FIGS. 10A, 108, 10C, 11A, 118, 11C, 22A and 22B, of securing the driving element in the driving element securing member of this disclosure will now be described in relation to FIG. 21. As described above, the driving element comprises an end portion and a body portion, the body portion having a cross-sectional area that is less than that of the end portion.

As described above, the end portion 902 of the driving element is inserted 53001 into the driving element securing member into a first opening (1103, 1203a, 1303a) of the driving element securing member 1100, 1200, through a cavity (1104, 1204, 1304) and into a second opening (1103a, 1203, 1303a) of the driving element securing member 1100, 1200, 1300. The first opening (1103, 1203a, 1303) may be an opening formed in an external face of the driving element securing member 1100, 1200, 1300, in some cases this external face is a side face 1108a, 1108b, 1108c, 1208a, 1208b, 1208c, 1308a, 1308d or an end face 1107, 1307 as described above in relation to FIG. 10A, 108, 10C, 11A, 11B, 11C, 22A and 22B. The end portion 902 is connected to the body portion 904 of the driving element and thus the body portion 904 will follow the route taken by the end portion 902, although the body portion 904 will be more flexible than the end portion 902 of the driving element.

In step S3003 the driving element is rotated to a final orientation. In rotating the driving element to a final orientation, the body portion 904 of the driving element will enter at least one channel 1106, 1206a, 1206b, 1306 that is in communication with the cavity 1104, 1204, 1304 and configured to open to an external face of the driving element securing member 1100, 1200, 1300.

The end portion 902 will be inserted 53001 into the driving element securing member 900, 1000, in a first orientation that is different from a desired orientation. The desired orientation may be thought of as the final orientation in which the length of the end portion 902 of the driving element is arranged in the longitudinal direction of the driving element securing member 1100, 1200, 1300. Furthermore, in the final orientation the first opening and the second opening are configured to have a length in a longitudinal direction (shown in FIG. 118 as direction P2) of the driving element securing member 1100, 1200 that is less than a length of the end portion 902 of the driving element in said longitudinal direction when the end portion 902 of the driving element is retained within the cavity 1104, 1204. The first orientation may be a direction that is not parallel to the longitudinal direction of the driving element securing member and the first opening. For the configuration of driving element securing member 1300, wherein the second opening 1303a may not have a length that is less than the length of the crimp 902 in the longitudinal direction, the same insertion process will occur, however the cavity 1304 may be the same size as the second opening 1303a and the crimp 902 may therefore be able to rotate within the cavity 1304 and the second opening 1303a at the same time such that the crimp 902 may be able to freely exit the second side surface 1308b of the driving element securing member 1300.

Optionally, prior to rotating the driving element to the final orientation (in step S3003), the driving element may be rotated from the first orientation to an intermediate orientation S3002. In the intermediate orientation the body portion 904 of the driving element enters a channel that is in communication with the cavity 1204 and configured to open to a side face of the driving element securing member 1200. In this intermediate orientation the end portion 902 of the driving element may rotate within the cavity 1204 in order to rotate from the intermediate orientation to the final orientation. The step S3002 of rotating the driving element to an intermediate orientation prior to rotating to a final orientation is a purely optional step that may not be necessary in some driving element securing member geometries, for example the geometry of the driving element securing member 1100 shown in FIGS. 10A, 108 and 10C does not require this intermediate step. The geometry of the driving element securing member 1200 shown in FIGS. 11A, 11B and 11C however, does require an intermediate orientation to be reached prior to rotating to the final orientation. This is because the driving element securing member 1200 geometry described in relation to FIGS. 11A, 11B and 11C requires two rotational movements in order to move from the first orientation of insertion to the final orientation.

When the optional intermediate orientation is present in the example shown in FIGS. 11A to 11C, and thus step S3002 is implemented, the first rotation S3002 from the first orientation to the intermediate orientation is in a plane that is perpendicular to the longitudinal direction of the driving element securing member 1200. This is a rotation that is about an axis that is centred along the longitudinal direction of the driving element securing member 1200 (an axis that runs through the driving element securing member 1200 in the plane P2). The rotation from the intermediate orientation to the final orientation is in a plane parallel to the longitudinal direction of the driving element securing member. In other words, the rotation of the driving element to the final orientation from the intermediate orientation is about an axis that is perpendicular to the longitudinal direction of the driving element securing member 1200. In this way, the end portion (crimp) 902 of the driving element can rotate in the cavity 1204 formed in the driving element securing member 1200 and the body portion 904 of the driving member can rotate from a first channel 1206b formed in a side face of the driving element securing member 1200 into a channel 1206a formed in an end face of the driving element securing member 1200.

When the geometry of the driving element securing member is such that only one rotational movement is necessary to move from a first orientation to the final orientation (e.g. with the driving element securing member 1100 shown in FIGS. 10A to 10C), the rotation is performed in one step S3003 (i.e. step S3002 is not performed). In this case, the rotation from the first orientation to the final orientation is in a plane parallel to the longitudinal direction of the driving element securing member 1100. In other words, the driving element may rotate in one movement by rotating about an axis that perpendicular to the longitudinal axis of the driving element securing member 1100 and in the plane P1. The end portion 902 of the driving element will rotate within the cavity 1104 formed in the driving element securing member 1100 and the body portion 904 of the driving element will rotate through the channel 1106 formed in the external face, which may be an end face, of the driving element securing member 1100 to reach the final orientation.

The final orientation of the driving element is that in which the length of the end portion 902 of the driving element is arranged in the longitudinal direction of the driving element securing member. Furthermore, in the final orientation the first opening and the second opening are configured to have a length in the longitudinal direction (shown in FIG. 11B as direction P2) of the driving element securing member 1100, 1200 that is less than a length of the end portion 902 of the driving element in said longitudinal direction when the end portion 902 of the driving element is retained within the cavity 1104, 1204. In the final orientation the body portion 904 and the end portion 902 of the driving element securing member 1100, 1200 are arranged along the longitudinal axis of the driving element securing member 1100, 1200.

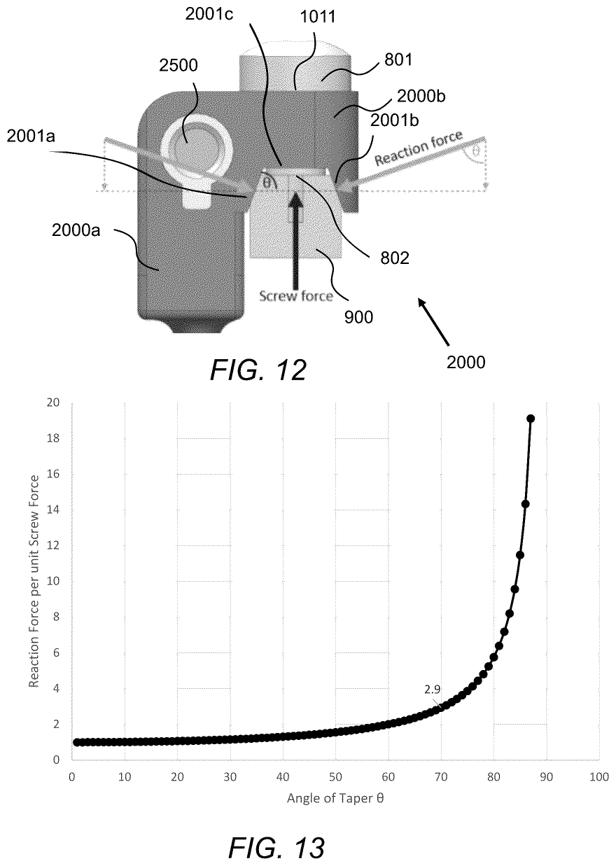

The interaction between the holding member 2000 and the driving element securing member 900 will now be described with reference initially to FIG. 12. The holding member 2000 of the present disclosure is configured to restrain the driving element securing member 900 from moving relative to it. To do this the holding member 2000 may be formed in a substantially/approximately upside down “L” shape. In other words, the holding member 2000 may have a first portion 2000a that extends transverse (e.g., perpendicular) to the longitudinal direction x of the shaft, and a second portion 2000b that extends away from one end of the first portion 2000a in a direction that is perpendicular both to a direction parallel to the longitudinal direction of the shaft and the direction in which the first portion 2000a extends. In some cases, the angle formed between the first portion 2000a and the second portion 2000b of the holding member 2000 may be greater than 90 degrees. For example, the inside angle of the “L” shape may be greater than 90 degrees if the second portion 2000b that extends away from one end of the first portion 2000a in a direction that is perpendicular to a direction parallel to the longitudinal direction of the shaft but is not perpendicular to the direction in which the first portion 2000a extends. The portion of the holding member 2000 at which the first and second portions 2000a, 2000b are connected comprises a through hole 2500 through which a rail of the instrument interface may be provided. This allows the holding member 2000 to slide along the rail to adjust the position of the holding member 2000 within the instrument interface. The holding member 2000 comprises at least one wall 2001 that defines a recess in the second portion 2000b. The surface of the second portion 2000b that extends away from the first portion 2000a of the holding member, and is configured to contact the drive element securing member 900, includes the at least one wall.

In the linear configuration the at least one wall may be comprised of two walls 2001a, 2001b that oppose/face each other and are connected by a third wall 2001c to form a recess in the second portion 2000b of the holding member 2000 (which may be referred to as a “fin lug”). In this configuration the third wall 2001c is parallel to the direction in which the second portion 2000b of the holding member 2000 extends. The third wall 2001c is perpendicular to the direction in which the driving element securing member 900 is received in the recess. The third wall 2001c partially extends along the length of the second portion 2000b of the holding member 2000. The third wall 2001c is connected at a first end by the first wall 2001a and at a second end by the second wall 2001b of the recess. In this configuration these three walls form the recess configured to receive the driving element securing member 900. The one or more wall 2001a may extend away from the third wall 2001c towards the driving element securing member 900 at an angle, α, that is greater than 90 degrees. In this case the first and second walls 2001a, 2001b extend away from the third wall 2001c at angles greater than 90 degrees, preferably between 95 and 125 degrees, more preferably between 105 and 115, most preferably 110 degrees. FIG. 12 shows a complimentary angle, θ, wherein θ=180−α degrees. The at least one wall 2001a (in this example two) that define the recess have a shape and/or angle that compliments the shape and/or angle of at least one tapered side wall 1109 (best seen in FIG. 10) of the driving element securing member 900, 1100, 1200. In other words, the at least one wall 2001a of the holding member 2000 that forms the recess is configured to mate with/contact at least one tapered side wall 1109 of the driving element securing member 900, 1100, 1200 once the driving element securing member 900 is received in the recess. The third wall 2001c may contact the driving element securing member 900 without any tapered surface whereas the at least one other wall 2001a, 2001b contacts the driving element securing member with a tapered surface.

As can be seen in FIG. 12, once the driving element securing member 900 is received in the recess, it is held in place by both frictional forces as well as the force applied by a fixing member 801. In the case of the example of FIG. 12 the fixing member 801 is a screw that passes through a second opening 1011 (through hole/slot) formed in the second portion 2000b of the holding member 2000 such that the fixing member 801 can pass completely through the holding member 2000. The fixing member 801 then enters the first opening 901 in the driving element securing member 900 and is secured to the driving element securing member 900. This may be achieved by a threaded portion that mates with the thread on the fixing member 801 in the case that it is a screw. In some instances, there may be a washer 802 between the third wall 2001c of the holding member 2000 and the driving element securing member 900. The fixing member 801 can then be fastened to retain the driving element securing member 900. When the driving element securing member 900 is received in the recess and the at least one (in this configuration two) tapered side wall 1109 of the driving element securing member contacts the at least one wall of the holding member 2001a, 2001b, a frictional force is present between the two surfaces. In addition, as the driving element securing member 900 is secured in the recess by the fixing member 801 the at least one tapered side wall 1109 will experience a reaction force caused by the tapered wall 1109 contacting the at least one wall 2001a of the holding member 2000. This reaction force increases as the angle theta between the third wall 2001c and the at least one of the first or second walls 2001a, 2000b, increases towards 90 degrees. This can also be seen in FIG. 12 as the angle between a horizontal plane in which the second portion extends away from the first portion 2000a of the holding member 2000 and the at least one wall (first or second). In other words, the at least one tapered side wall is tapered from the direction perpendicular to the direction of in which the first portion of the holding member extends by a taper angle of between 60 degrees and 90 degrees, preferably 70 degrees as shown in FIG. 13. Using complementary tapered side walls as in this configuration, it is possible to increase the frictional force on the driving element securing member, by approximately 50%, using the same force applied by the screw (screw force). As such, it is possible to maintain the same frictional force on the driving element securing member by tapering the side walls and decreasing the screw force shown in FIG. 12.

Increasing the frictional force will be beneficial in terms of retaining the driving element securing member in the holding member, and slippage of the driving element securing member from its intended position in the holding member is prevented or reduced. A component of the reaction force, that is exerted in response to the vertical screw force, is exerted in the opposite direction to the vertical screw force. The reaction force is applied by the at least one wall of the holding member against the at least one tapered side wall of the driving element securing member. The angle of the reaction force changes with the angle (theta θ) shown in FIG. 13. This equation represents an ideal approximation of the relationship between the reaction force and the angle θ. The magnitude of the reaction force changes according to the following expression.

$$R = \frac{{R}_{f}}{cos θ}$$

where R is the reaction force at the side walls (2001a and 2001b), R_{f} is the reaction force for a flat surface (i.e. for θ=0), and θ is the angle as described above and shown in FIGS. 12 and 13. The reaction force per unit of screw force increases as the taper angle increases. When the walls of the recess of the holding member 2000 are not angled and are parallel to the direction in which the first portion 2000a of the holding member 2000 extends (i.e. θ is 90 degrees), no reaction force is exerted by the walls as at this angle only one flat plane is present that is perpendicular to the direction of the screw force. The reaction force exerted by this wall (2001c) is therefore equal and opposite to the screw force. The graph of FIG. 13 shows how the reaction force at the side walls (2001a and 2001b) per unit screw force changes with the taper angle. As shown in the figure, when the taper angle is 0 degrees cos θ is 1 and from the equation above the reaction force at the side walls (2001a and 2001b) is equal to the reaction force of a flat surface. In other words, the reaction force is equal and opposite to the screw force shown in FIG. 12 such that the two have a one to one relationship. In this case there is no recess only a planar surface that extends perpendicular to the first portion of the holding member. Conversely, when the taper angle is 90 degrees cos θ is 0, such that the graph in FIG. 13 appears to go to infinity (due to the divide by zero in the equation above), but in this case since the side walls are parallel to the screw force there is no reaction force perpendicular to the side walls 2001a and 2001b. In this scenario, the walls 2001a and 2001b do not exert a force on the driving element securing member and only the wall 2001c applies a force that opposes the screw force. In this case the reaction force is exerted by the third wall 2001c. Thus the driving element securing member is not prevented from sliding by the walls 2001a and 2001b and is only retained in the recess by the screw. The frictional force may be given by the expression,

F=μR

Where F is the frictional force, R is the reaction force and μ is the static friction coefficient. The static friction coefficient will depend on the materials used and the surface properties of parts. The materials, manufacturing methods and finish/coatings of the parts will define the friction coefficient.

The advantage of using tapered/angled walls for the holding member 1000 and/or driving element securing member 900, 1100, 2100 is that it increases the retention of the cable 904 (held by the driving element securing member 900, 1100, 2100) in position without changing the input force applied by the fixing member 801 that connects the holding member 1000, 2000 and the driving element securing member 900, 1100, 2100 (i.e. without increasing the torque at which the screw 801 is tightened, or the screw and driving element securing member 900, 1100, 2100 dimensions).

The following describes steps to be carried out during manufacture following assembly of the instrument in order to set the tension of the driving elements and the alignment of the instrument interface elements.

First, the instrument interface elements are set to the alignment position. For example, if the alignment position is with each instrument interface element at the mid-point of its travel over its displacement range, then the instrument interface elements are aligned to these positions. Next, the end effector is placed in the predetermined configuration. Next, the pairs of driving elements are tensioned. This may be done using any of the tensioning mechanisms described herein, for example by sliding a tensioning pulley along a rail or through a socket, or by displacing a pair of lug elements (drive element securing members).

Once tensioned, the displacement position of the instrument interface element is then set to the predetermined alignment position using the alignment mechanism. For example, in the implementation shown in FIGS. 7A, 7B and 7C, the screw 1940, 1941 is loosened, and the body 1933, 1935 of the instrument interface element displaced along the rail 1930, 1929 relative to the lug 1938, 1937 until the body of the instrument interface element is in the predetermined alignment position. The screw is then tightened. The screw 1940, 1941 and the lug 1938, 1937 in this example are the same as the fixing member 801 and the drive element securing member 900, 1100, 2100 described above.

An instrument interface as above but having a rotational configuration will now be described. The below described instrument interfaces and drive assembly interface examples are suitable for being attached to the terminal end of the robot arm described above and for driving each of the described instruments via its engagement with the corresponding instrument interface. Each of the described instrument interfaces is suitable for being attached to the proximal end of any of the instruments described herein and for driving articulation of the end effector of the instrument via the described driving elements when driven itself via its engagement with the corresponding drive assembly interface.

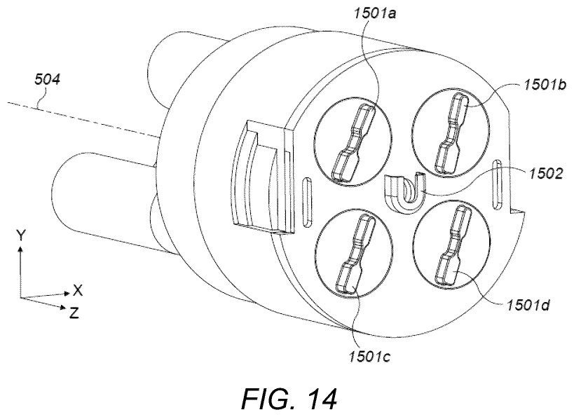

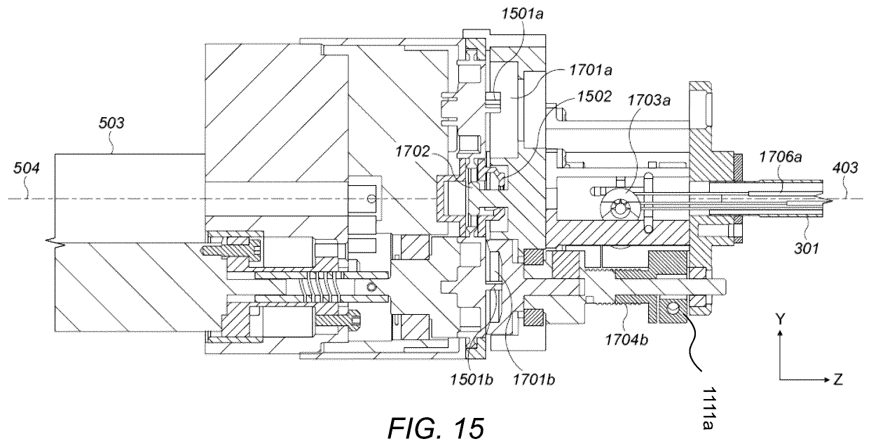

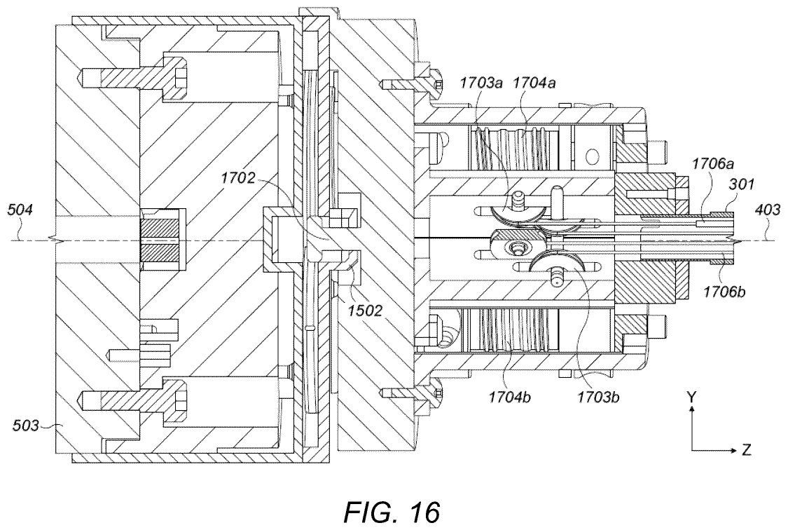

FIGS. 14 to 16 illustrate an example in which a rotary interface is used (rather than a linear interface as in the previous examples). In particular, FIG. 14 shows a perspective view of a drive assembly interface of a terminal link of a robot arm; and FIGS. 15 and 16 show cross-sectional views of the drive assembly interface being connected to an instrument interface of an instrument. Common reference numerals are used in FIGS. 14 to 16 to denote the same components in the different figures. Each of FIGS. 14 to 16 shows the direction of the X, Y and Z axes, wherein the Z direction is along the longitudinal axis of the terminal link and along the longitudinal axis of the shaft of the instrument, and wherein the Y direction is the detachment direction. The drive assembly interface comprises four driving elements: 1501a, 1501b, 1501c and 1501d. In this example, the driving elements are rotary driving elements which can rotate about respective axes parallel to the longitudinal axis 504 of the terminal link (i.e. they can rotate about respective axes which are parallel to the Z direction). As in the examples described above, the driving elements are arranged in a plane which is perpendicular to the longitudinal axis 504 of the terminal link (which is parallel to the Z direction). In particular, the driving elements are arranged in a plane which is parallel to the direction of detachment (i.e. along the Y direction).

Each of the drive assembly interface elements (1501a, 1501b, etc.) engages with a corresponding instrument interface element (1701a, 1701b, etc.) of an instrument interface of an instrument (shown in FIGS. 15 to 16). The arm can cause the drive assembly interface elements 1501 to rotate, which in turn causes the instrument interface elements 1701 to rotate. In this way, rotational force (i.e. torque) can be transferred over the interface from the drive assembly Interface elements to the instrument interface elements. The instrument interface is attached to the shaft 301 of the instrument. Each instrument interface element comprises a capstan 1704 (which may be referred to as a “spindle”), to which it is attached, and around which driving elements 1706 can be wound. The driving elements 1706a, 1706b are for driving one or more joints of the articulation of the instrument. In this example, the driving elements (1706a, 1706b) are flexible cables allowing them to be wound around the capstans (1704a, 1704b). Each of the driving elements 1706 is arranged to move around a respective redirecting pulley 1703 and through the shaft 301 of the instrument. When an instrument interface element 1701 is rotated, the corresponding capstan 1704 rotates which causes the corresponding driving element 1706 to move through the shaft 301 in a direction parallel to the longitudinal axis 403 of the shaft. The capstans of the rotary interface may be thought of in the same terms as the holding member and the driving element securing member of the linear instrument interface described above.

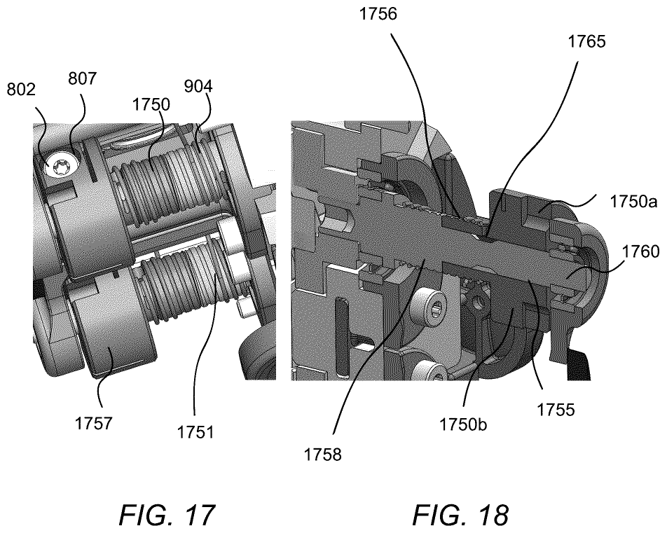

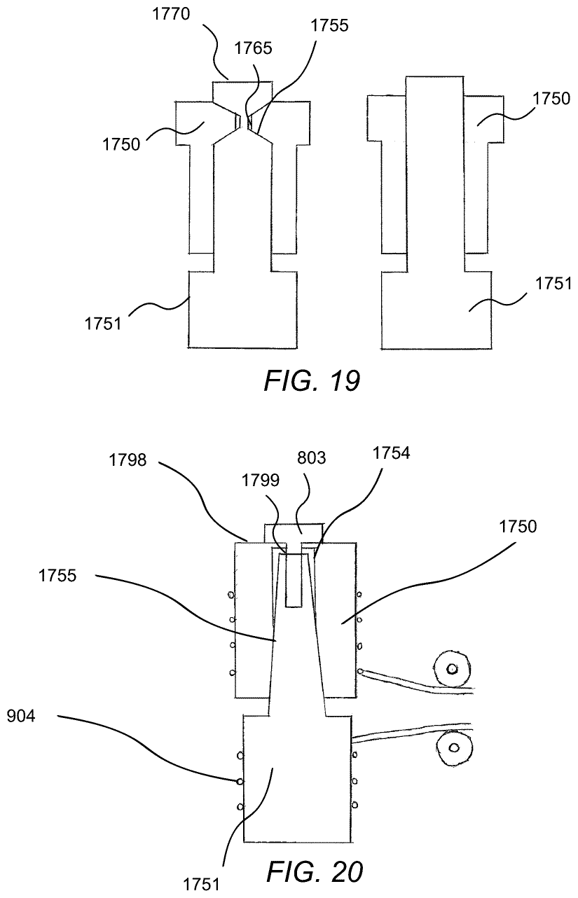

The configuration of the capstans will now be described in detail in relation to FIG. 17 as well as FIGS. 17-20. The capstan shown in FIGS. 18, 19 and 20 (1704b) may be comprised of at least two parts. A bottom/first capstan block 1750 (a holding member) and a top/second capstan block 1751 (a driving element securing member) as shown in the cross-section view of FIGS. 15 and 18. As can be seen in the figures, the top capstan block 1751 has an elongate body that has a cylindrical cross section, a portion of the which may have a cable channel formed on the outer surface. This cable channel is configured to be coupled to at least one driving element 904, which may be wound around the cable channel. In other words, the at least one driving element may, at one end, be connected to top and/or bottom capstan block and be wound around the cable channel in the top and/or bottom capstan block. This end portion 902 may be coupled to the top and/or bottom capstan block. This configuration allows the at least one driving element to be wound and unwound from either capstan block. The top capstan block 1751 then narrows in diameter in the longitudinal direction most proximal to the end effector, such that it may be received by the bottom capstan block. The narrower portion 1760 of the top capstan block may have at least one tapered wall (contact surface) 1755 e.g., the outer surface of the narrower portion that is received in the bottom capstan block 1750. The narrower portion may be thought of as a second portion (e.g., non-cable channel portion) and may be cylindrical in shape. The full length of the at least one wall 1755 of the narrower portion 1760 of the top capstan block 1751 may be tapered uniformly or the at least one wall 1755 of the top capstan block 1751 may be tapered as shown in FIGS. 18 and 19 such that only a portion of the at least one wall 1755 is tapered to form at least one groove 1765. The at least one wall 1755, may also have a plurality of grooves arranged around the circumference of at least one wall 1755. These plurality of grooves 1765 may be equally spaced or may be randomly spaced along the narrower portion 1760. The grooves may also be formed at multiple positions along the second portion (narrower portion) of the top capstan block. In other words, the narrower portion 1760 (e.g., non-cable channel portion) of the top capstan block may have a non-cylindrical shape along its length in some examples. The narrower portion 1760 (e.g., non-cable channel portion) of the top capstan block 1751 that is received in the bottom capstan block 1750 may be uniformly cylindrical and the at least one wall may be straight (extending parallel to the longitudinal direction of the shaft) with the exception of at least one groove 1765 formed in the at least one wall 1755. The groove 1765 may extend around the circumference of the narrow portion 1760 of the top capstan block. This can be seen in the cross-section view of FIGS. 18 and 19. There may also be a plurality or at least one groove 1765 formed around/extend around the circumference of the narrow portion 1760 of the top capstan block. These may be formed at various positions along the longitudinal direction of the top capstan block. In some cases, the end surface 1770 of the substantially cylindrical top capstan block 1751 that is most proximal to the end effector of the instrument may comprise a first opening (not shown) that is configured to receive a fixing member 803. In some instances the fixing member 803 may be a screw or equivalent member.

The instrument interface element comprised of the capstan 1704b also comprises the bottom capstan block 1750 e.g., the holding member, that is configured to form or contain a recess 1754 to receive the top capstan block 1751. As can be seen in the figures, in particular FIG. 20, the bottom capstan block 1750 has an elongate body that has a cylindrical cross section, a portion of the which may have a cable channel formed on the outer surface, shown in FIGS. 20 and 21. This cable channel is configured to be fast with at least one driving element, which may be wound around the cable channel. This can be best seen in FIG. 17. The bottom capstan block 1750 is shaped to have a cable channel portion 1756 and a head portion 1757, the head portion 1757 having a diameter that is larger than the diameter of the cable channel portion 1756. The cable channel portion 1756 of the bottom capstan block 1750 may be the same diameter as the cable channel portion 1758 of the top capstan block 1751 and the two cable channel portions 1756 and 1758 may abut one another. In some cases, the bottom capstan block 1750 may be comprised of more than one portion, in particular two half capstan portions 1750a, 1750b, a first and second. The two portions 1750a, 1750b of the of the bottom capstan block 1750 may be formed along an axis parallel to axis 504 shown in FIG. 15. The bottom capstan block 1750 comprises a recess 1754, which may be a through hole, within which the narrower portion 1760 of the top capstan block 1751 is received. The through hole may be formed of a first through hole portion formed in the first half capstan block 1750a and a second through hole portion formed in the second half capstan block 1750b. The diameter of the recess 1754 of the bottom capstan block 1750 and the diameter of the non-cable channel portion (narrower portion) 1760 of the top capstan block 1751 are configured to be fast with each other. The recess 1754 of the bottom capstan block 1750 having at least one wall 1765 that is shaped to compliment the shape of the at least one tapered side wall 1755 (of the non-cable channel portion) of the top capstan block 1751. In the configuration in which the bottom capstan block 1750 is comprised of more than one portion, each portion 1750a, 1750b forms a section of the recess 1754 such that when the portions are joined together, they form a recess 1754 within which the top capstan block 1751 can be received. This recess 1754 surrounds the circumference of at least a non-cable channel portion (the narrow portion) 1760 of the top capstan block 1751 as can be seen in FIGS. 18 to 20. The more than one portion of the bottom capstan block 1750 may be secured to each other using a fixing member 802 that enters an opening 807 in one portion 1750a of the bottom capstan block 1750 and is received by an opening (not shown) in a second portion 1750b of the bottom capstan block 1750, this is shown from a plan view in FIG. 17. The one or more portions of the bottom capstan block may not contact each other when they are secured together by the fixing member 802 and may be prevented from contacting each other by the diameter/cross-section of the portion of the top capstan block which they surround. In this way the at least one fixing member 802 may connect the portions of the bottom capstan block 1750 together. The fixing member 802 may be tightened and loosened by the user to apply different levels of force between the portions of the bottom capstan block 1750. This allows the portions of the bottom capstan block 1750 to securely enclose at least a portion 1760 of the top capstan block 1751 gripping the top capstan block 1751 and ensuring the top capstan block 1751 and the bottom capstan block 1750 are fast with each other. In some configurations, the bottom capstan block 1750 may be a single part and not comprised of more than one portion. In this case, the bottom capstan block 1750 may optionally include a deformable portion 1111a shown in FIG. 15. This deformable portion 1111a is configured to deflect towards the top capstan block and is capable of deforming towards the top capstan block when a fixing member that passes through the deformable portion 1111a is tightened. This will cause the deformable portion 1111a, which may also be known as a flange, to deflect from a first position, in which it does not contact the top capstan block, to a second position, in which it does contact the top capstan block. The bottom capstan block may include more than one deformable portion.

When the top capstan block 1751 and the bottom capstan block 1750 are fast with one another they will rotate simultaneously, therefore winding or unwinding the driving elements 904. This allows the position of the end effector to be zeroed. The fixing member 802 that holds the portions of the bottom capstan block together may be loosened of release the pressure applied by the bottom capstan block 1750 to the top capstan block 1751 that has been received within its recess 1754. In this configuration the inner surfaces 1765 of the recess 1754 formed by the bottom capstan block 1750 will apply less pressure to the outer surface 1755 of the narrower portion of the top capstan block 1751 and the top and bottom capstan blocks 1750, 1751 will no longer be fast with one another. This will allow the two capstan blocks 1750, 1751 to be rotated independently of one another during use for winding or unwinding the driving elements that are coupled to the capstan blocks and are wound around the cable channel portions formed on the outer surfaces of each capstan block. This allows the driving elements to be independently tensioned. In an alternate configuration the head portion 1757 of the bottom capstan block 1750 may include a first opening configured to receive a further fixing member. The first opening being a through hole in the head portion 1757 of the bottom capstan block 1750 that opens on one side to the outside of the bottom capstan block 1750 and opening on the other side to the inside of the recess 1754 in the bottom capstan block 1750 to contact the top capstan block 1751 received therein. In this way when the fixing member is tightened and protrudes further into the recess it applies a force to the outer surface of the narrow portion 1760 of the top capstan block 1751. The force applied to the outer surface 1765 of the narrow portion 1760 of the top capstan block 1751 is increased such that the top and bottom capstan blocks 1750, 1751 become fast with one another.

As seen in the cross-sectional views of FIGS. 19 and 20, the bottom capstan block 1750 may have at least one wall 1765 that defines the recess 1754 being tapered or angled in order to complement the shape of the tapered side walls 1755 of the top capstan block 1751 that is received in the recess 1757. The at least one wall 1765 of the bottom capstan block 1750 may include protrusions that mate with the groove formed by the tapered side walls 1755 of the top capstan block as shown in FIG. 19. FIG. 19 illustrates a configuration in which the recess 1757 formed in the bottom capstan block 1750 is a through hole that surrounds the top capstan block 1751 and includes protrusions that complement the openings formed in the narrower portion 1760 of the top capstan block. An alternate configuration is shown in FIG. 20 wherein the bottom capstan block 1750 surrounds and at least partially covers the end surface 1799 of the top capstan block 1751 most proximal to the instrument end effector. In this configuration the portion 1760 of the top capstan block 1751 that is received in the recess 1757 of the bottom capstan block 1750 terminates inside the recess 1757. A fixing member may be inserted through an opening 803 (second opening) in the end surface 1798 of the bottom capstan block 1750 most proximal to the instrument end effector and be received in an opening in the end surface 1799 of the top capstan block 1751 that is most proximal to the instrument end effector. The fixing member 803 applies a force between the capstan block portions to secure the top capstan block 1751 within the recess 1757 of the bottom capstan block 1750 such that the at least one wall 1765 of the bottom capstan block 1750 is in frictional contact with the at least one tapered side wall 1755 of the top capstan block 1751.

In the examples shown in FIGS. 14 to 16, rotational drive from a motor is transformed into rotation of the driving elements around the capstan. This can be a more efficient system for transferring force from the drive assembly of the robot arm to the end effector than a system in which rotational drive is transformed into linear drive across the interface.

The instrument could be used for non-surgical purposes. For example, it could be used in a cosmetic procedure.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description, it will be evident to a person skilled in the art that various modifications may be made within the scope of the invention.

1. A robotic surgical instrument comprising:
an articulation for articulating an end effector, the articulation driveable by at least one driving element; and
an instrument interface comprising an instrument interface element for driving the at least one driving element, the instrument interface element movable over a range, the at least one driving element coupled to the instrument interface element such that movement of the instrument interface element is transferred to the at least one driving element;
wherein the instrument interface element comprises:
a driving element securing member comprising at least one tapered side wall and configured to be coupled to the at least one driving element;
a fixing member; and
a holding member that includes an opening configured to receive the fixing member and at least one wall defining a recess having a shape that is complementary to the shape of the at least one tapered side wall of the driving element securing member;
wherein the fixing member is configured to be received in the opening and apply force to secure the driving element securing member within the recess of the holding member so that the at least one wall of the holding member is in frictional contact with the tapered side wall of the driving element securing member.

2. The robotic surgical instrument according to claim 1, wherein the driving element securing member further comprises an opening configured to receive the fixing member; and
wherein the fixing member is configured to be received in the opening of the driving element securing member and the opening of the holding member.

3. The robotic surgical instrument according to claim 1, wherein the recess includes at least two tapered side walls; and the at least two tapered side walls oppose each other.

4. The robotic surgical instrument according to claim 1, wherein the driving element securing member is a cable end block, and
wherein a first one of the at least two driving elements is configured to terminate in the cable end block and a second one of the at least two of driving elements is configured to terminate in the cable end block.

5. The robotic surgical instrument as claimed in claim 1, wherein the robotic surgical instrument comprises a tensioning mechanism for tensioning the at least one driving element; and
wherein the tensioning mechanism comprises a screw adjustment mechanism which couples a pair of drive element securing members together for linearly displacing the pair of drive element securing members with respect to each other.

6. The robotic surgical instrument as claimed in claim 5, wherein the robotic surgical instrument further comprises an alignment mechanism for setting the displacement position of the instrument interface element to a predetermined alignment position when the end effector has a predetermined configuration; and
wherein the screw adjustment mechanism comprises a screw captive in the first drive element securing member and constrained by the first drive element securing member so as to prevent the screw from displacing linearly with respect to the first drive element securing member, the screw being threaded through the second drive element securing member, thereby causing the drive element securing members to displace linearly towards each other on the screw being tightened and to displace linearly away from each other on the screw being loosened.

7. The robotic surgical instrument as claimed in claim 1, wherein the instrument interface element is linearly displaceable along a displacement axis parallel to a longitudinal axis of a shaft of the instrument.

8. The robotic surgical instrument as claimed in claim 7, wherein the displacement axis is offset from the longitudinal axis of the shaft.

9. The robotic surgical instrument as claimed in claim 1, wherein the holding member is displaceable linearly between a minimum displacement position and a maximum displacement position,
wherein a pair of driving elements are coupled to with the driving element securing member, the driving element securing member being linearly displaceable within the holding member, and
wherein the driving element securing member is linearly displaceable along a driving element securing member axis which is parallel to the axis along which the holding member is linearly displaceable.

10. The robotic surgical instrument as claimed in claim 9, wherein an alignment mechanism comprises a screw adjustment mechanism coupled to the holding member and driving element securing member for adjusting the displacement position of the holding member without displacing the driving element securing member.

11. The robotic surgical instrument as claimed in claim 10, wherein the screw adjustment mechanism comprises a screw threaded into the driving element securing member through a slot in the holding member, the slot being aligned with the driving element securing member axis, the screw being constrained to slide along the slot, thereby permitting the holding member to be displaced relative to the driving element securing member when the screw is loose, and causing the holding member to be held fast with the driving element securing member when the screw is tight.

12. The robotic surgical instrument according to claim 1, wherein the holding member is a first capstan block that is configured to be rotatable;
the driving element securing member is a second capstan block that is configured to be rotatable; and
wherein the recess is a through hole in the first capstan block.

13. The robotic surgical instrument according to claim 12, wherein the first capstan block comprises:
a first half capstan block; and
a second half capstan block;
wherein the first and second half capstan blocks are configured to surround the driving element securing member.

14. The robotic surgical instrument according to claim 13, wherein the through hole is formed of a first through hole portion formed in the first half capstan block and a second through hole portion formed in the second half capstan block.

15. The robotic surgical instrument according to claim 12, wherein the first capstan block is configured to rotate relative to the second capstan block to tension the at least one driving element.

16. The robotic surgical instrument according to claim 12, wherein the first capstan block and the second capstan block are configured to rotate in unison to change the offset of the end effector; and
wherein there are at least two driving elements, a first driving element configured to wrap around the first capstan block and a second driving element configured to wrap around the second capstan blocks.

17. The robotic surgical instrument according to claim 12, wherein the through hole in the first capstan block includes at least one tapered side wall.

18. The robotic surgical instrument according to claim 13, wherein each of the first half capstan block and the second half capstan block include a tapered side wall.

19. The robotic surgical instrument according to claim 17, wherein the second capstan block includes at least one tapered side wall configured to contact the at least one tapered side wall of the first capstan block.

20. The robotic surgical instrument according to claim 1, wherein the at least one tapered side wall of the driving element securing member is tapered from the direction perpendicular to the direction in which the drive element securing member is received in the holding member by a taper angle of between 60 degrees and 90 degrees.
